(19) United States
(12) Patent Application Publication
(10) Pub. No.: US 2025/0231195 A1
(43) Pub. Date: Jul. 17, 2025

(54) USE OF FSH-RECEPTOR LIGANDS FOR DIAGNOSIS AND THERAPY OF ATHEROSCLEROSIS

(71) Applicant: FSHR THERANOSTICS, Paris (FR)

(72) Inventor: Nicolae GHINEA, Paris (FR)

(73) Assignee: FSHR THERANOSTICS, Paris (FR)

(21) Appl. No.: 18/294,707

(22) PCT Filed: Aug. 4, 2022
(86) PCT No.: PCT/EP2022/071961

§ 371 Date: Feb. 2, 2024

(30) Foreign Application Priority Data
Aug. 4, 2021 (EP) 21306087.4

(51) Int. Cl. G01N 33/68; A61K 38/24; A61K 39/00; C07K 14/59; C07K 16/28

(52) U.S. Cl. CPC G01N 33/6893; A61K 38/24; C07K 14/59; C07K 16/2869; A61K 2039/505; G01N 2333/726; G01N 2800/323

(57) ABSTRACT

Diagnostic imaging and therapy of active atherosclerotic lesions. More particularly, ligands that specifically target the follicle-stimulating hormone receptor (FSHR) expressed by cells associated with the atherosclerotic plaques including the arterial endothelial cells covering the plaques, endothelial cells of vasa vasorum supplying arteries affected by atherosclerosis, macrophages, macrophage-derived giant cells, and foam cells.

### FIELD OF INVENTION

The present invention relates to the diagnostic imaging and therapy of active atherosclerotic lesions. More particularly, ligands that specifically target the follicle-stimulating hormone receptor (FSHR) expressed by cells associated with the atherosclerotic plaques including the arterial endothelial cells covering the plaques, endothelial cells of vasa vasorum supplying arteries affected by atherosclerosis, macrophages, macrophage-derived giant cells, and foam cells.

### BACKGROUND OF INVENTION

Atherosclerosis is a focal disease process that may result from either a lipid disorder (dyslipidemia) and/or an inflammatory process, having as ultimate out-come, the atherosclerotic plaque, a lesion located within the intima of large- and medium-sized arteries in regions with disturbed blood flow (arches, branches, and bifurcations). The rupture of the atherosclerotic plaques is a major cause of myocardial infarction and stroke. Eighty percent of cardiovascular deaths (i.e., each year 14.3 million people die globally) are due to heart attacks and strokes, and one third of these deaths occur prematurely in people under 70 years of age. Therefore, identification and differentiation of vulnerable plaques at high risk of thromboembolic events are important in all vascular beds to intensify and specify selection criteria for primary and secondary treatment.

Various complementary non-invasive imaging modalities including B-mode ultrasound of the neck for evaluation of carotid artery intima-media thickness or plaque, aortic and carotid magnetic resonance imaging (MRI), computed tomography (CT) of the chest for evaluation of coronary artery calcification, single photon emission computed tomography (SPECT), and positron emission tomography (PET) can be used for detection and quantification of atherosclerosis through its stages in different vascular beds.

Although very sensitive, PET has poor spatial resolution, and therefore, integrated PET scanners are used to co-register PET images with CT or MRI for accurate anatomical localization. Anatomical evaluation of the atherosclerotic plaques considering only the degree of luminal stenosis overlooks features associated with vulnerable plaques, such as high-risk morphological features or pathophysiology, and hence risks missing vulnerable or ruptured non-stenotic plaques.

Currently, PET imaging with ¹⁸F-fluorodeoxyglucose (FDG) provides a useful marker of atherosclerotic lesions, in particular the inflammation activity in the atherosclerotic plaques. However, this tracer lacks inflammatory cell specificity and, therefore, is not a practical solution for imaging the coronary vasculature because of high background myocardial signal. To overcome these limitations, novel PET tracers that can more accurately identify individual cell components of plaques are necessary. Consequently, there is interest in identifying the markers of vulnerability using MRI for morphology and PET for physiological processes involved in atherogenesis.

In the present invention, the Inventor discloses the identification of the presence of FSHR in endothelial cells, macrophages, and foam cells in atherosclerotic plaques of patients, which has never been disclosed in the prior art. More particularly, the Inventor presents evidence that FSHR, which is absent in normal arterial tissue, is expressed in the atherosclerotic plaques in arteries affected by atherosclerosis and demonstrates that the FSHR expression in the atherosclerotic lesions increases with the advancement of the disease. This is the first marker of atherosclerotic plaques that is exposed on the luminal surface of the endothelial cells in direct contact with blood. Therefore, the use of FSHR ligands (including the monoclonal antibodies anti-hFSHR and FSH) coupled to imaging agents (labeled with radioisotopes or fluorochromes) and therapeutic agents intravenously injected offers unique opportunities for screening, diagnosis, prevention, and treatment of high-risk plaques occurring in vulnerable patients.

### SUMMARY

The present invention relates to a follicle-stimulating hormone receptor (FSHR) ligand for use as an imaging agent for imaging or diagnosing a condition associated with an atherosclerotic lesion.

In one embodiment, the FSHR ligand is detectably labeled FSH.

In one embodiment, the FSHR ligand is a detectably labeled FSHR-binding aptamer.

In one embodiment, the FSHR ligand is a detectably labeled anti-FSHR antibody or antigen-binding fragment thereof.

In one embodiment, the FSHR ligand is a detectably labeled anti-FSHR antibody or antigen-binding fragment thereof comprising:

(a) a variable region of the heavy chain
comprising the following CDRs:
(SEQ ID NO: 1)
VH-CDR1: QFYVG,
(SEQ ID NO: 6)
VH-CDR2: EIFPRTGNTNYNEKFKG,
(SEQ ID NO: 11)
VH-CDR3: GPTASGYAMDY,
and
a variable region of the light chain comprising
the following CDRs:
(SEQ ID NO: 12)
VL-CDR1: RSSQSIVHRNGNTYLE,
(SEQ ID NO: 13)
VL-CDR2: KVSNRFS,
and
(SEQ ID NO: 14)
VL-CDR3: FQGSHVPFT;
or
(b) a variable region of the heavy chain
comprising the following CDRs:
(SEQ ID NO: 2)
VH-CDR1: RQWVI,
(SEQ ID NO: 7)
VH-CDR2: EILPRNGNTNYNEKFKG,
(SEQ ID NO: 11)
VH-CDR3: GPTASGYAMDY,
and
a variable region of the light chain comprising
the following CDRs:
(SEQ ID NO: 12)
VL-CDR1: RSSQSIVHRNGNTYLE,
(SEQ ID NO: 13)
VL-CDR2: KVSNRFS,
and
(SEQ ID NO: 14)
VL-CDR3: FQGSHVPFT;
or
(c) a variable region of the heavy chain
comprising the following CDRs:
(SEQ ID NO: 3)
VH-CDR1: KQWLL,
(SEQ ID NO: 8)
VH-CDR2: EIFPRNGNTNYNEKFKG,
(SEQ ID NO: 11)
VH-CDR3: GPTASGYAMDY,
and
a variable region of the light chain comprising
the following CDRs:
(SEQ ID NO: 12)
VL-CDR1: RSSQSIVHRNGNTYLE,
(SEQ ID NO: 13)
VL-CDR2: KVSNRFS,
and
(SEQ ID NO: 14)
VL-CDR3: FQGSHVPFT;
or
(d) a variable region of the heavy chain
comprising the following CDRs:
(SEQ ID NO: 4)
VH-CDR1: RSWIL,
(SEQ ID NO: 9)
VH-CDR2: EIYPQNQNTNYNEKFKG,
(SEQ ID NO: 11)
VH-CDR3: GPTASGYAMDY,
and
a variable region of the light chain comprising
the following CDRs:
(SEQ ID NO: 12)
VL-CDR1: RSSQSIVHRNGNTYLE,
(SEQ ID NO: 13)
VL-CDR2: KVSNRFS,
and
(SEQ ID NO: 14)
VL-CDR3: FQGSHVPFT;
or
(e) a variable region of the heavy chain
comprising the following CDRs:
(SEQ ID NO: 5)
VH-CDR1: KYWTQ,
(SEQ ID NO: 10)
VH-CDR2: EIYPRNGNTNYNEKFKG,
(SEQ ID NO: 11)
VH-CDR3: GPTASGYAMDY,
and
a variable region of the light chain comprising
the following CDRs:
(SEQ ID NO: 12)
VL-CDR1: RSSQSIVHRNGNTYLE,
(SEQ ID NO: 13)
VL-CDR2: KVSNRFS,
and
(SEQ ID NO: 14)
VL-CDR3: FQGSHVPFT.

In one embodiment, the FSHR ligand is a detectably labeled anti-FSHR antibody or antigen-binding fragment thereof comprising:

(a) a heavy chain comprising the amino acid
sequence:
(SEQ ID NO: 15)
QVQLQQSGAELMKPGASVKISCKATGYTFSQFYVGWVKQRPGHGLEWIGE
IFPRTGNTNYNEKFKGKATFTADTSSSTAYMQLSSLTSEDSAVYYCARGP
TASGYAMDYWGQGTSVTVSS,
and
a light chain comprising the amino acid sequence:
(SEQ ID NO: 20)
DVLMTQTPLSLPVSLGDQASISCRSSQSIVHRNGNTYLEWYLQKPGQSPK
LLIYKVSNRFSGVPDRFSGSGSGTDFTLKIIRVEAEDLGVYYCFQGSHVP
FTFGSGTKLEIK;
or
(b) a heavy chain comprising the amino acid
sequence:
(SEQ ID NO: 16)
QVQLQQSGAELMKPGASVKISCKATGYTFSRQWVIWVKQRPGHGLEWIGE
ILPRNGNTNYNEKFKGKATFTADTSSSTAYMQLSSLTSEDSAVYYCARGP
TASGYAMDYWGQGTSVTVSS,
and
a light chain comprising the amino acid sequence:
(SEQ ID NO: 20)
DVLMTQTPLSLPVSLGDQASISCRSSQSIVHRNGNTYLEWYLQKPGQSPK
LLIYKVSNRFSGVPDRFSGSGSGTDFTLKIIRVEAEDLGVYYCFQGSHVP
FTFGSGTKLEIK;
or
(c) a heavy chain comprising the amino acid
sequence:
(SEQ ID NO: 17)
QVQLQQSGAELMKPGASVKISCKATGYTFSKQWLLWVKQRPGHGLEWIGE
IFPRNGNTNYNEKFKGKATFTADTSSSTAYMQLSSLTSEDSAVYYCARGP
TASGYAMDYWGQGTSVTVSS,
and
a light chain comprising the amino acid sequence:
(SEQ ID NO: 20)
DVLMTQTPLSLPVSLGDQASISCRSSQSIVHRNGNTYLEWYLQKPGQSPK
LLIYKVSNRFSGVPDRFSGSGSGTDFTLKIIRVEAEDLGVYYCFQGSHVP
FTFGSGTKLEIK;
or
(d) a heavy chain comprising the amino acid
sequence:
(SEQ ID NO: 18)
QVQLQQSGAELMKPGASVKISCKATGYTFSRSWILWVKQRPGHGLEWIGE
IYPQNQNTNYNEKFKGKATFTADTSSSTAYMQLSSLTSEDSAVYYCARGP
TASGYAMDYWGQGTSVTVSS,
and
a light chain comprising the amino acid sequence:
(SEQ ID NO: 20)
DVLMTQTPLSLPVSLGDQASISCRSSQSIVHRNGNTYLEWYLQKPGQSPK
LLIYKVSNRFSGVPDRFSGSGSGTDFTLKIIRVEAEDLGVYYCFQGSHVP
FTFGSGTKLEIK;
or
(e) a heavy chain comprising the amino acid
sequence:
(SEQ ID NO: 19)
QVQLQQSGAELMKPGASVKISCKATGYTFSKYWTQWVKQRPGHGLEWIGE
IYPRNGNTNYNEKFKGKATFTADTSSSTAYMQLSSLTSEDSAVYYCARGP
TASGYAMDYWGQGTSVTVSS,
and
a light chain comprising the amino acid sequence:
(SEQ ID NO: 20)
DVLMTQTPLSLPVSLGDQASISCRSSQSIVHRNGNTYLEWYLQKPGQSPK
LLIYKVSNRFSGVPDRFSGSGSGTDFTLKIIRVEAEDLGVYYCFQGSHVP
FTFGSGTKLEIK.

In one embodiment, the FSHR ligand is in form of an imaging composition for intravenous administration.

The present invention further relates to a method for collecting imaging data on the atherosclerotic plaques, wherein said method comprises:
a) providing an imaging agent comprising a FSHR ligand as described herein;
b) administering said imaging agent to a patient; and
c) collecting imaging data in said patient.

The present invention further relates to a FSHR blocking agent for use for treating atherosclerosis in a subject in need thereof.

In one embodiment, the FSHR blocking agent is an agent that blocks FSHR expression or intracellular signaling in atherosclerotic plaques.

In one embodiment, the FSHR blocking agent is an anti-FSHR antibody or antigen-binding fragment thereof that prevents FSH induced signaling in atherosclerotic plaques.

In one embodiment, the FSHR blocking agent is an anti-FSHR antibody or antigen-binding fragment thereof comprising:

(a) a variable region of the heavy chain
comprising the following CDRs:
(SEQ ID NO: 1)
VH-CDR1: QFYVG,
(SEQ ID NO: 6)
VH-CDR2: EIFPRTGNTNYNEKFKG,
(SEQ ID NO: 11)
VH-CDR3: GPTASGYAMDY,
and
a variable region of the light chain comprising
the following CDRs:
(SEQ ID NO: 12)
VL-CDR1: RSSQSIVHRNGNTYLE,
(SEQ ID NO: 13)
VL-CDR2: KVSNRFS,
and
(SEQ ID NO: 14)
VL-CDR3: FQGSHVPFT;
or
(b) a variable region of the heavy chain
comprising the following CDRs:
(SEQ ID NO: 2)
VH-CDR1: RQWVI,
(SEQ ID NO: 7)
VH-CDR2: EILPRNGNTNYNEKFKG,
(SEQ ID NO: 11)
VH-CDR3: GPTASGYAMDY,
and
a variable region of the light chain comprising
the following CDRs:
(SEQ ID NO: 12)
VL-CDR1: RSSQSIVHRNGNTYLE,
(SEQ ID NO: 13)
VL-CDR2: KVSNRFS,
and
(SEQ ID NO: 14)
VL-CDR3: FQGSHVPFT;
or
(c) a variable region of the heavy chain
comprising the following CDRs:
(SEQ ID NO: 3)
VH-CDR1: KQWLL,
(SEQ ID NO: 8)
VH-CDR2: EIFPRNGNTNYNEKFKG,
(SEQ ID NO: 11)
VH-CDR3: GPTASGYAMDY,
and
a variable region of the light chain comprising
the following CDRs:
(SEQ ID NO: 12)
VL-CDR1: RSSQSIVHRNGNTYLE,
(SEQ ID NO: 13)
VL-CDR2: KVSNRFS,
and
(SEQ ID NO: 14)
VL-CDR3: FQGSHVPFT;
or
(d) a variable region of the heavy chain
comprising the following CDRs:
(SEQ ID NO: 4)
VH-CDR1: RSWIL,
(SEQ ID NO: 9)
VH-CDR2: EIYPQNQNTNYNEKFKG,
(SEQ ID NO: 11)
VH-CDR3: GPTASGYAMDY,
and
a variable region of the light chain comprising
the following CDRs:
(SEQ ID NO: 12)
VL-CDR1: RSSQSIVHRNGNTYLE,
(SEQ ID NO: 13)
VL-CDR2: KVSNRFS,
and
(SEQ ID NO: 14)
VL-CDR3: FQGSHVPFT;
or
(e) a variable region of the heavy chain
comprising the following CDRs:
(SEQ ID NO: 5)
VH-CDR1: KYWTQ,
(SEQ ID NO: 10)
VH-CDR2: EIYPRNGNTNYNEKFKG,
(SEQ ID NO: 11)
VH-CDR3: GPTASGYAMDY,
and
a variable region of the light chain comprising
the following CDRs:
(SEQ ID NO: 12)
VL-CDR1: RSSQSIVHRNGNTYLE,
(SEQ ID NO: 13)
VL-CDR2: KVSNRFS,
and
(SEQ ID NO: 14)
VL-CDR3: FQGSHVPFT.

In one embodiment, the FSHR blocking agent is an anti-FSHR antibody or antigen-binding fragment thereof comprising:

(a) a heavy chain comprising the amino acid
sequence:
(SEQ ID NO: 15)
QVQLQQSGAELMKPGASVKISCKATGYTFSQFYVGWVKQRPGHGLEWIGE
IFPRTGNTNYNEKFKGKATFTADTSSSTAYMQLSSLTSEDSAVYYCARGP
TASGYAMDYWGQGTSVTVSS,
and
a light chain comprising the amino acid sequence:
(SEQ ID NO: 20)
DVLMTQTPLSLPVSLGDQASISCRSSQSIVHRNGNTYLEWYLQKPGQSPK
LLIYKVSNRFSGVPDRFSGSGSGTDFTLKIIRVEAEDLGVYYCFQGSHVP
FTFGSGTKLEIK;
or
(b) a heavy chain comprising the amino acid
sequence:
(SEQ ID NO: 16)
QVQLQQSGAELMKPGASVKISCKATGYTFSRQWVIWVKQRPGHGLEWIGE
ILPRNGNTNYNEKFKGKATFTADTSSSTAYMQLSSLTSEDSAVYYCARGP
TASGYAMDYWGQGTSVTVSS,
and
a light chain comprising the amino acid sequence:
(SEQ ID NO: 20)
DVLMTQTPLSLPVSLGDQASISCRSSQSIVHRNGNTYLEWYLQKPGQSPK
LLIYKVSNRFSGVPDRFSGSGSGTDFTLKIIRVEAEDLGVYYCFQGSHVP
FTFGSGTKLEIK;
or
(c) a heavy chain comprising the amino acid
sequence:
(SEQ ID NO: 17)
QVQLQQSGAELMKPGASVKISCKATGYTFSKQWLLWVKQRPGHGLEWIGE
IFPRNGNTNYNEKFKGKATFTADTSSSTAYMQLSSLTSEDSAVYYCARGP
TASGYAMDYWGQGTSVTVSS,
and
a light chain comprising the amino acid sequence:
(SEQ ID NO: 20)
DVLMTQTPLSLPVSLGDQASISCRSSQSIVHRNGNTYLEWYLQKPGQSPK
LLIYKVSNRFSGVPDRFSGSGSGTDFTLKIIRVEAEDLGVYYCFQGSHVP
FTFGSGTKLEIK;
or
(d) a heavy chain comprising the amino acid
sequence:
(SEQ ID NO: 18)
QVQLQQSGAELMKPGASVKISCKATGYTFSRSWILWVKQRPGHGLEWIGE
IYPQNQNTNYNEKFKGKATFTADTSSSTAYMQLSSLTSEDSAVYYCARGP
TASGYAMDYWGQGTSVTVSS,
and
a light chain comprising the amino acid sequence:
(SEQ ID NO: 20)
DVLMTQTPLSLPVSLGDQASISCRSSQSIVHRNGNTYLEWYLQKPGQSPK
LLIYKVSNRFSGVPDRFSGSGSGTDFTLKIIRVEAEDLGVYYCFQGSHVP
FTFGSGTKLEIK;
or
(e) a heavy chain comprising the amino acid
sequence:
(SEQ ID NO: 19)
QVQLQQSGAELMKPGASVKISCKATGYTFSKYWTQWVKQRPGHGLEWIGE
IYPRNGNTNYNEKFKGKATFTADTSSSTAYMQLSSLTSEDSAVYYCARGP
TASGYAMDYWGQGTSVTVSS,
and
a light chain comprising the amino acid sequence:
(SEQ ID NO: 20)
DVLMTQTPLSLPVSLGDQASISCRSSQSIVHRNGNTYLEWYLQKPGQSPK
LLIYKVSNRFSGVPDRFSGSGSGTDFTLKIIRVEAEDLGVYYCFQGSHVP
FTFGSGTKLEIK.

### Definitions

In the present invention, the following terms have the following meanings:

The terms “antibody” and “immunoglobulin” include antibodies or immunoglobulins of any isotype, fragments of antibodies which retain specific binding to antigen, including, but not limited to, Fab, Fab′, F(ab′)2, Fv, scFv, Fd, diabodies, single domain antibodies (sdAbs), linear antibodies, single-chain antibody molecules, and multispecific antibodies formed from antibody fragments, chimeric antibodies, humanized antibodies, single-chain antibodies, and fusion proteins comprising an antigen-binding portion of an antibody and a non-antibody protein. Antibodies can be polyclonal or monoclonal, multiple or single chain, or intact immunoglobulins, and may be derived from natural sources or from recombinant sources. Antibodies can be tetramers of immunoglobulin molecules. The basic 4-chain antibody unit is a heterotetrameric glycoprotein composed of two identical light (L) chains and two identical heavy (H) chains. In the case of IgGs, the 4-chain unit is generally about 150,000 daltons. Each L chain is linked to a H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the α and γ chains and four CH domains for μ and ε isotypes. Each L chain has at the N-terminus, a variable domain (VL) followed by a constant domain (CL) at its other end. The VL is aligned with the VH and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, CT, 1994, page 71 and Chapter 6. The L chain from any vertebrate species can be assigned to one of two clearly distinct types, called kappa and lambda, based on the amino acid sequences of their constant domains. Depending on the amino acid sequence of the constant domain of their heavy chains (CH), immunoglobulins can be assigned to different classes or isotypes. The five classes of immunoglobulins: IgA, IgD, IgE, IgG, and IgM have heavy chains designated α, δ, ε, γ, and μ, respectively. The γ and α classes are further divided into subclasses on the basis of relatively minor differences in CH sequence and function, e.g., humans express the following subclasses: IgG1, IgG2, IgG3, IgG4, IgA1, and IgA2.

As intended herein an “antigen-binding fragment” relates to an antibody fragment which retains its specific binding properties towards FSHR according to the invention. Such fragments notably encompass Fab fragments (which can be produced by papain cleavage of antibodies), F(ab′)2 fragments (which can be produced by pepsin cleavage of antibodies) or Fab′ fragments (which can be produced by pepsin cleavage of antibodies followed by a reducing treatment).

The term “atherosclerosis” as used herein refers to a chronic lipid-driven inflammatory disease of the arteries that is characterized by, for example, the accumulation of lipids within the arterial wall and the formation of an atherosclerotic plaque (atheroma or fibroinflammatory lipid plaque) in the vessel wall of medium- or large-sized elastic or muscular arteries, thereby impairing arterial function and resulting in ischemia or infarction of the heart, brain, or extremities when obstructing the lumen of the main arteries (upon complete stenosis or plaque rupture). “Atherosclerotic lesions” develop at sites of endothelial injury and are located overlying fatty streaks and diffuse intimal thickening, an intimal change thought to be a physiological adaptation to mechanical stress and present in atherosclerotic-prone areas of the artery including branch points, branch ostia, and curvatures. Hemodynamic shear stress regulates pro- and antiatherogenic genes and their gene products in the vascular wall cells. Endothelial injury, inflammation, lipid metabolism, thrombosis, and tissue injury and repair are processes involved in preclinical atherosclerotic plaque buildup. Transition from preclinical to symptomatic clinical atherosclerosis occurs when the plaque encroaches significantly into the lumen or when an acute thrombotic event occurs, most often due to plaque rupture, resulting in flow obstruction within the artery lumen. Atherosclerosis results in acute or chronic ischemic injury to the organ supplied by the diseased artery and can be a silent killer as it leads to acute coronary syndrome including myocardial infarction and to stroke.

As intended herein an “aptamer” relates to a nucleic acid or peptide molecule, in particular a ribonucleic acid molecule. Aptamers are a class of molecule that represents an alternative to antibodies in term of molecular recognition. Aptamers are oligonucleotide or oligopeptide sequences with the capacity to recognize virtually any class of target molecules with high affinity and specificity.

The term “diagnosis” means the identification of the disease or the assessment of the severity of the disease.

As used herein, the terms “follicle-stimulating hormone receptor” or “FSHR” refer to the receptor to which the methods of the invention apply. The follicle stimulating hormone receptor or FSH-receptor (FSHR) is a transmembrane receptor that interacts with the follicle stimulating hormone (FSH) and represents a G protein-coupled receptor (GPCR). As used herein, the term “FSHR” also denotes the antigen recognized by the anti FSHR antibodies and in particular the products of the FSHR gene.

The follicle stimulating hormone, a central hormone of mammalian reproduction, is produced mainly in the anterior pituitary gland and the classical target organs are the ovary and testis. In females, FSH stimulates follicular maturation and estrogen production through aromatization of androgens. In males, FSH functions such as stimulation of Sertoli cell proliferation in immature testis and maintenance of qualitatively and quantitatively normal spermatogenesis have been proposed. FSH exerts its biological role by binding to the plasma membrane FSHR. FSHR is known to be mainly expressed by testicular Sertoli cells, and ovarian granulosa cells (Sprengel et al., 1990, Simoni et al., 1997), as well as in the female reproductive tract (Stilley et al., 2016). FSHR expression was observed within endothelial cells of blood vessels associated with various pathological conditions (ex., cancer [Radu et al., 2010], benign prostatic hyperplasia [Radu et al. 2010], and endometriosis [Robin et al., 2016; Ponikwicka-Tyszko et al., 2016]. An exemplary of the amino-acid sequence of the FSHR is available in the UniProtKB/Swiss-Protdatabase under the accession number: P23945.

The terms “follicle-stimulating hormone receptor ligand” or “FSHR ligand” refer to any compound liable to specifically bind to FSHR as defined above. A ligand can thus comprise or can consist of one or several binding moieties. In particular, when a ligand comprises one or several binding moieties, it can also comprise at least one “detectable marker”, that is a moiety the presence of which can be readily detected according to methods well known to the person skilled in the art.

Preferably, in the ligands according to the invention, at least one binding moiety is specific for FSHR. Also, preferably at least one binding moiety is selected from the group consisting of FSH, an antibody, an antigen-binding antibody fragment, a single-chain variable antibody fragment (scFv), and an aptamer. Methods for producing an antibody, an antigen-specific antibody fragment, a scFv, or an aptamer are well-known to the person skilled in the art.

The term “FSHR blocking agent” refers to any compound which inhibits or suppresses the expression or activity of the receptor. It is preferably an anti-FSHR antibody. In another particular embodiment, it can be a siRNA, or an antisense molecule. It may also be a chemical agent or a peptide. More preferably the FSHR ligand or blocking agent is a monoclonal antibody against FSHR.

“Framework” or “FR” residues are those variable domain residues other than the hypervariable region residues herein defined.

The term “heavy chain” refers to the larger of the two types of polypeptide chains present in antibody molecules in their naturally occurring conformations, and which normally determines the class to which the antibody belongs.

The terms “hypervariable region” and “complementarity determining region” and their respective abbreviations (HVR, HV, CDR) are used interchangeably herein. Further, the following pairs of terms are also used interchangeably herein:
“VH CDR1” and “HCDR1”;
“VH CDR2” and “HCDR2”;
“VH CDR3” and “HCDR3”;
“VL CDR1” and “LCDR1”;
“VL CDR2” and “LCDR2”; and
“VL CDR3” and “LCDR3”.

The term “light chain” refers to the smaller of the two types of polypeptide chains present in antibody molecules in their naturally occurring conformations. Kappa (K) and lambda (λ) light chains refer to the two major antibody light chain isotypes.

A “scFv” relates to a single-chain variable fragment of an antibody, that is an immunoglobulin short chain variable region and an immunoglobulin large chain variable region linked together by a peptide.

The term “variable” refers to the fact that certain segments of the variable domains differ extensively in sequence among antibodies. The V domain mediates antigen binding and defines specificity of a particular antibody for its particular antigen. However, the variability is not evenly distributed across the 110- to 130-amino acid span of the variable domains. Instead, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, MD. (1991)).

The “variable region” or “variable domain” of an antibody refers to the amino-terminal domains of the heavy or light chain of the antibody. The variable domain of the heavy chain may be referred to as “VH”, “VH” or “H”. The variable domain of the light chain may be referred to as “VL”, “VL” or “L”. These domains are generally the most variable parts of an antibody and contain the antigen-binding sites.

The term “prognosis” means the assessment of the outcome of the condition, i.e., to determine the evolution of the condition, and the risk of worsening.

The term “subject” as used herein refers to an animal. Preferably the animal is a mammal. More preferably the mammal is a human. A subject can be male or female. In one embodiment, a subject may be a “patient”, i.e., a warm-blooded animal, more preferably a human, who/which is awaiting the receipt of, or is receiving medical care or was/is/will be the object of a medical procedure, or is monitored for the development of a specific disease or condition. The term “subject”, is intended for a human or non-human mammal affected or likely to be affected with a condition associated with atherosclerosis. Said patient is preferably a human being.

The terms “treating” or “treatment” refer to both therapeutic treatment and prophylactic or preventative measures; wherein the object is to prevent, reduce, alleviate, and/or slow down (lessen) one or more symptoms of atherosclerosis in a subject in need thereof. Symptoms of atherosclerosis include, without being limited to, chest pain or angina, shortness of breath, fatigue, confusion, limb pain, muscle weakness. In one embodiment, “treating” or “treatment” refers to a therapeutic treatment. In another embodiment, “treating” or “treatment” refers to a prophylactic or preventive treatment. In yet another embodiment, “treating” or “treatment” refers to both a prophylactic (or preventive) treatment and a therapeutic treatment. Those in need of treatment include those already with the disorder as well as those prone to have the disorder or those in whom the disorder is to be prevented. A subject or mammal is successfully “treated” for a disease or condition if, after receiving a therapeutic amount of a therapeutic agent, the patient shows observable and/or measurable reduction in or absence of one or more of the following: relief to some extent, of one or more of the symptoms associated with atherosclerosis; reduced morbidity and mortality, and improvement in quality-of-life issues. The above parameters for assessing successful treatment and improvement in the disease are readily measurable by routine procedures familiar to a physician.

### DETAILED DESCRIPTION

The present invention first relates to a FSHR ligand for use as an imaging agent, preferably for use for imaging or diagnosing a condition associated with an atherosclerotic lesion.

More particularly, the present invention provides a FSH receptor (FHSR) ligand, for use as an imaging agent for imaging or diagnosing an atherosclerotic lesion, by detecting expression of FSHR in vessels, macrophages, giant cells, foam cells associated with said atherosclerotic lesion.

Imaging or diagnosing a condition associated with an atherosclerotic lesion or an atherosclerotic plaque can be performed in vivo, in vitro, or ex vivo.

The FSHR ligand is advantageously useful for localizing or determining the size of an atherosclerotic plaque, or for evaluating the severity of an atherosclerotic lesion or monitoring the efficacy of an anti-atherosclerosis therapy.

The invention provides an imaging agent that is designed to target an atherosclerotic lesion in a mammal, and which can be detected following its administration to the mammalian body in vivo by imaging procedures e.g., PET. The imaging agent consists of a FSHR ligand, which may be detectably labeled with a detectable molecule or substance, such as a fluorescent molecule, a radioactive molecule or any other labels known in the art to provide (either directly or indirectly) a signal.

The present invention thus provides an imaging agent for use in an in vivo diagnostic or imaging method, e.g., Single Photon Emission Computed Tomography (SPECT) or Positron Emission Tomography (PET).

The invention provides an imaging agent that is designed to target an atherosclerotic lesion in a sample from a mammal, and which can be detected by in vitro or ex vivo imaging procedures e.g., microscopy. The imaging agent consists of a FSHR ligand, which may be detectably labeled with a detectable molecule or substance, such as a fluorescent molecule, a radioactive molecule, or any other labels known in the art to provide (either directly or indirectly) a signal.

The present invention thus provides an imaging agent for use in an in vitro or ex vivo diagnostic or imaging method, for example and without limitation, fluorescent microscopy, electron microscopy, or immunohistochemistry,

Another object of the invention is a method for collecting imaging data, wherein said method comprises:
a) providing an imaging agent comprising a detectably labeled FSH receptor ligand;
b) administering a patient with said imaging agent;
c) collecting imaging data in said patient.

Another object of the invention is a method for diagnosing a condition associated with atherosclerosis, wherein said method comprises:
a) providing an imaging agent comprising a detectably labeled FSHR ligand;
b) administering a patient with said imaging agent;
c) collecting imaging data in said patient;
d) detecting the atherosclerotic plaques and diagnosing the condition associated with the atherosclerotic plaques.

In a preferred embodiment, the imaging agent of step a) is radioactively labeled FSH or a radioactively labeled anti-FSHR antibody or antigen-binding fragment thereof.

The present invention further provides a method for monitoring the efficacy of an anti-atherosclerosis agent (unrelated to FSH or FSHR) by sequential imaging of the atherosclerotic plaque size using a FSHR ligand.

Examples of the atherosclerotic therapies include but are not limited to statins and radiotherapy.

This aspect of the invention relates to methods of determining the efficacy of agents for treating atherosclerosis in a subject who has been treated with an agent, by detecting the expression of FSHR in cells and blood vessels, in particular in microvessels, associated with atherosclerotic plaques. The expression of FSHR can be detected by any of the methods described above using the imaging agent of the invention.

The level of FSHR expression which is utilized as a diagnostic marker for drug efficacy can be determined by using the imaging agent in the same subject prior to and after drug treatment. A compelling difference is significant of the drug achieving its effect. For example, successful hormonal treatment of atherosclerosis is expected to be accompanied by loss or strong diminution of FSHR expression in the atherosclerotic plaques, which can be detected using the imaging method of the invention.

Another object of the invention is a FSH receptor (FSHR) blocking agent, preferably an anti-FSHR antibody or antigen-binding fragment, for use in the treatment of a condition associated with an atherosclerotic lesion.

In one embodiment the FSHR blocking agent is an anti-FSHR antibody or antigen-binding fragment that prevents FSH induced signaling in atherosclerotic plaques.

In one embodiment the FSHR blocking agent is an agent that blocks FSHR expression or intracellular signaling in atherosclerotic plaques.

In one embodiment, the FSHR ligand targets atherosclerotic lesions in a mammal, and can be detected following its administration in vivo, e.g., by SPECT or PET.

The invention further relates to a method for the treatment of atherosclerosis, by intravenous delivery of a FSHR blocking agent.

Administration of FSHR ligand or blocking agent as an imaging agent or as a pharmaceutical composition in therapy may be advantageously performed by intravenous administration.

In one embodiment, the FSHR ligand may be the Follicle Stimulating Hormone (FSH), like human FSH or recombinant FSH, preferably recombinant human FSH, produced in CHO cells or in bacteria. Bacterially expressed FSH is not glycosylated but may maintain the ability to bind to FSHR, and therefore could induce fewer side effects in its physiological target organs, the testicles and the ovaries. Derivatives of FSH are further encompassed, e.g., deglycosylated FSH or a peptide fragment derived from the FSH sequence.

The human FSH typically refers to the heterodimeric protein constituted of the “follicle-stimulating hormone alpha chain” also named “glycoprotein hormones alpha chain” or “FSH-alpha”, referenced as P01215 in the UniProtKB/Swiss-Prot database, and of the “follicle-stimulating hormone beta subunit” also named “follitropin subunit beta” or “FSH-beta”, referenced as P01225 in the UniProtKB/Swiss-Prot database. The reference follicle-stimulating hormone alpha chain human protein corresponds to SEQ ID NO: 27, and the reference follicle-stimulating hormone beta subunit human protein corresponds to SEQ ID NO: 28.

In one embodiment the FSHR ligand may be the human Follicle Stimulating Hormone (FSH) or an active fragment or derivative thereof. In one embodiment the FSHR ligand may be the human follicle-stimulating hormone alpha chain, such as sequence SEQ ID NO: 27. In one embodiment the FSHR ligand may be the human follicle-stimulating hormone beta subunit, such as sequence SEQ ID NO: 28.

In another embodiment, the FSHR ligand is detectably labeled FSH, preferably detectably labeled recombinant human FSH or an active fragment or derivative thereof. In one embodiment the FSHR ligand may be a detectably labeled human follicle-stimulating hormone alpha chain, such as sequence SEQ ID NO: 27. In one embodiment the FSHR ligand may be a detectably labeled human follicle-stimulating hormone beta subunit, such as sequence SEQ ID NO: 28.

Also included within the scope of the invention is a FSHR ligand as described in WO2018/069831. In one embodiment the FSHR ligand may be the human follicle-stimulating hormone beta subunit, such as sequence SEQ ID NO: 29. In one embodiment the FSHR ligand may be a detectably labeled human follicle-stimulating hormone beta subunit, such as sequence SEQ ID NO: 29.

As used herein, the term “labeled”, with regard to the FSHR ligand, such as an antibody or FSH, is intended to encompass direct labeling by coupling (i.e., physically linking) a detectable substance, such as a radioactive agent or a near-infrared fluorophore (e.g., indocyanine green (ICG)) to the ligand, as well as indirect labeling by reactivity with a detectable substance. A FSHR ligand may be labeled with a radioactive molecule by any method known in the art. For example, radioactive molecules include but are not limited to radioactive atoms for scintigraphy studies such as 123I, 124I, 111In, 186Re, 188Re.

In one embodiment, the FSHR ligand is a FSHR-binding chemical agent. In a particular embodiment, the FSHR ligand is a detectably labeled FSHR-binding chemical agent.

In one embodiment, the FSHR ligand is a FSHR-binding peptide. In another particular embodiment, the FSHR ligand is a detectably labeled FSHR-binding peptide.

In one embodiment, the FSHR ligand is a FSHR-binding aptamer. In another particular embodiment, the FSHR ligand is a detectably labeled FSHR-binding aptamer.

In one embodiment, the FSHR ligand is an anti-FSHR antibody or antigen-binding fragment thereof. In one embodiment, the FSHR ligand is an anti-FSHR antibody or antigen-binding fragment thereof, preferably the monoclonal anti-human FSH antibody FSHR-323. In one embodiment, the FSHR ligand is a detectably labeled anti-FSHR antibody or antigen-binding fragment thereof.

Also included within the scope of the invention are FSHR antibodies or antigen-binding fragments as described in WO2018/172078. In one embodiment the anti-FSHR antibody or antigen-binding fragment thereof comprises:

(a) a variable region of the heavy chain
comprising the following CDRs:
(SEQ ID NO: 1)
VH-CDR1: QFYVG,
(SEQ ID NO: 6)
VH-CDR2: EIFPRTGNTNYNEKFKG,
and
(SEQ ID NO: 11)
VH-CDR3: GPTASGYAMDY,
and
(b) a variable region of the light chain
comprising the following CDRs:
(SEQ ID NO: 12)
VL-CDR1: RSSQSIVHRNGNTYLE,
(SEQ ID NO: 13)
VL-CDR2: KVSNRFS,
and
(SEQ ID NO: 14)
VL-CDR3: FQGSHVPFT.

In one embodiment the anti-FSHR antibody or antigen-binding fragment thereof comprises:

(a) a variable region of the heavy chain
comprising the following CDRs:
(SEQ ID NO: 2)
VH-CDR1: RQWVI,
(SEQ ID NO: 7)
VH-CDR2: EILPRNGNTNYNEKFKG,
and
(SEQ ID NO: 11)
VH-CDR3: GPTASGYAMDY,
and
(b) a variable region of the light chain
comprising the following CDRs:
(SEQ ID NO: 12)
VL-CDR1: RSSQSIVHRNGNTYLE,
(SEQ ID NO: 13)
VL-CDR2: KVSNRFS,
and
(SEQ ID NO: 14)
VL-CDR3: FQGSHVPFT.

In one embodiment the anti-FSHR antibody or antigen-binding fragment thereof comprises:

(a) a variable region of the heavy chain
comprising the following CDRs:
(SEQ ID NO: 3)
VH-CDR1: KQWLL,
(SEQ ID NO: 8)
VH-CDR2: EIFPRNGNTNYNEKFKG,
and
(SEQ ID NO: 11)
VH-CDR3: GPTASGYAMDY,
and
(b) a variable region of the light chain
comprising the following CDRs:
(SEQ ID NO: 12)
VL-CDR1: RSSQSIVHRNGNTYLE,
(SEQ ID NO: 13)
VL-CDR2: KVSNRFS,
and
(SEQ ID NO: 14)
VL-CDR3: FQGSHVPFT.

In one embodiment the anti-FSHR antibody or antigen-binding fragment thereof comprises:

(a) a variable region of the heavy chain
comprising the following CDRs:
(SEQ ID NO: 4)
VH-CDR1: RSWIL,
(SEQ ID NO: 9)
VH-CDR2: EIYPQNQNTNYNEKFKG,
and
(SEQ ID NO: 11)
VH-CDR3: GPTASGYAMDY,
and
(b) a variable region of the light chain
comprising the following CDRs:
(SEQ ID NO: 12)
VL-CDR1: RSSQSIVHRNGNTYLE,
(SEQ ID NO: 13)
VL-CDR2: KVSNRFS,
and
(SEQ ID NO: 14)
VL-CDR3: FQGSHVPFT.

In one embodiment the anti-FSHR antibody or antigen-binding fragment thereof comprises:

(a) a variable region of the heavy chain
comprising the following CDRs:
(SEQ ID NO: 5)
VH-CDR1: KYWTQ,
(SEQ ID NO: 10)
VH-CDR2: EIYPRNGNTNYNEKFKG,
and
(SEQ ID NO: 11)
VH-CDR3: GPTASGYAMDY,
and
(b) a variable region of the light chain
comprising the following CDRs:
(SEQ ID NO: 12)
VL-CDR1: RSSQSIVHRNGNTYLE,
(SEQ ID NO: 13)
VL-CDR2: KVSNRFS,
and
(SEQ ID NO: 14)
VL-CDR3: FQGSHVPFT.

In one embodiment the anti-FSHR antibody or antigen-binding fragment thereof comprises:

(a) a heavy chain comprising the amino acid
sequence:
(SEQ ID NO: 15)
QVQLQQSGAELMKPGASVKISCKATGYTFSQFYVGWVKQRPGHGLEWIGE
IFPRTGNTNYNEKFKGKATFTADTSSSTAYMQLSSLTSEDSAVYYCARGP
TASGYAMDYWGQGTSVTVSS,
and
(b) a light chain comprising the amino acid
sequence:
(SEQ ID NO: 20)
DVLMTQTPLSLPVSLGDQASISCRSSQSIVHRNGNTYLEWYLQKPGQSPK
LLIYKVSNRFSGVPDRFSGSGSGTDFTLKIIRVEAEDLGVYYCFQGSHVP
FTFGSGTKLEIK.

In one embodiment the anti-FSHR antibody or antigen-binding fragment thereof comprises:

(a) a heavy chain comprising the amino acid
sequence:
(SEQ ID NO: 16)
QVQLQQSGAELMKPGASVKISCKATGYTFSRQWVIWVKQRPGHGLEWIGE
ILPRNGNTNYNEKFKGKATFTADTSSSTAYMQLSSLTSEDSAVYYCARGP
TASGYAMDYWGQGTSVTVSS,
and
(b) a light chain comprising the amino acid
sequence:
(SEQ ID NO: 20)
DVLMTQTPLSLPVSLGDQASISCRSSQSIVHRNGNTYLEWYLQKPGQSPK
LLIYKVSNRFSGVPDRFSGSGSGTDFTLKIIRVEAEDLGVYYCFQGSHVP
FTFGSGTKLEIK.

In one embodiment the anti-FSHR antibody or antigen-binding fragment thereof comprises:

(a) a heavy chain comprising the amino acid
sequence:
(SEQ ID NO: 17)
QVQLQQSGAELMKPGASVKISCKATGYTFSKQWLLWVKQRPGHGLEWIGE
IFPRNGNTNYNEKFKGKATFTADTSSSTAYMQLSSLTSEDSAVYYCARGP
TASGYAMDYWGQGTSVTVSS,
and
(b) a light chain comprising the amino acid
sequence:
(SEQ ID NO: 20)
DVLMTQTPLSLPVSLGDQASISCRSSQSIVHRNGNTYLEWYLQKPGQSPK
LLIYKVSNRFSGVPDRFSGSGSGTDFTLKIIRVEAEDLGVYYCFQGSHVP
FTFGSGTKLEIK.

In one embodiment the anti-FSHR antibody or antigen-binding fragment thereof comprises:

(a) the heavy chain comprising the amino acid
sequence:
(SEQ ID NO: 18)
QVQLQQSGAELMKPGASVKISCKATGYTFSRSWILWVKQRPGHGL
EWIGEIYPQNQNTNYNEKFKGKATFTADTSSSTAYMQLSSLTSED
SAVYYCARGPTASGYAMDYWGQGTSVTVSS,
and
(b) the light chain comprising the amino acid
sequence:
(SEQ ID NO: 20)
DVLMTQTPLSLPVSLGDQASISCRSSQSIVHRNGNTYLEWYLQKP
GQSPKLLIYKVSNRFSGVPDRFSGSGSGTDFTLKIIRVEAEDLGV
YYCFQGSHVPFTFGSGTKLEIK

In one embodiment the anti-FSHR antibody or antigen-binding fragment thereof comprises:

(a) the heavy chain comprising the amino acid
sequence:
(SEQ ID NO: 19)
QVQLQQSGAELMKPGASVKISCKATGYTFSKYWTQWVKORPGHGL
EWIGEIYPRNGNTNYNEKFKGKATFTADTSSSTAYMQLSSLTSED
SAVYYCARGPTASGYAMDYWGQGTSVTVSS,
and
(b) the light chain comprising the amino acid
sequence:
(SEQ ID NO: 20)
DVLMTQTPLSLPVSLGDQASISCRSSQSIVHRNGNTYLEWYLQKP
GQSPKLLIYKVSNRFSGVPDRFSGSGSGTDFTLKIIRVEAEDLGV
YYCFQGSHVPFTFGSGTKLEIK

Also included within the scope of the invention are FSHR antibodies or antigen-binding fragments as described in WO2018/022505. In one embodiment, the anti-FSHR antibody or antigen-binding fragment thereof comprises:
(i) a heavy chain CDR1 (VH-CDR1) comprising SEQ ID NO: 30,
(ii) a heavy chain CDR2 (VH-CDR2) comprising SEQ ID NO: 31,
(iii) a heavy chain CDR3 (VH-CDR3) comprising SEQ ID NO: 32,
(iv) a light chain CDR1 (VL-CDR1) comprising SEQ ID NO: 33,
(v) a light chain CDR2 (VL-CDR2) comprising SEQ ID NO: 34, and
(vi) a light chain CDR3 (VL-CDR3) comprising SEQ ID NO: 35.

In one embodiment, the anti-FSHR antibody or antigen-binding fragment thereof comprises:

(i)
a heavy chain comprising the amino acid
sequence:
(SEQ ID NO: 36)
DVHLQESGPGLVKPSQSLSLTCTVTGFSITSDYAWNWIRQFPGNK
LEWMGSIFSSGSINYNPSLKSRISITRDTSRNQFFLQLNSVTTAD
AGTYYCARGGTGTDYWGQGTTLTVSS,
and
(ii)
a light chain comprising the amino acid
sequence:
(SEQ ID NO: 37)
DIQMTQTTSSLSASLGDRVTISCRASQDISNYLSWYQQKPDGTIK
LLIYYTSRLHSGVSSRFSGSGSGTDYSLTISNLEQEDFATYFCQQ
GHTLPPTFGGGTKLEIK

Also included within the scope of the invention are FSHR antibodies or antigen-binding fragments as described in WO2020/033797. In one embodiment, the anti-FSHR antibody or antigen-binding fragment thereof comprises:
(i) a heavy chain CDR1 (VH-CDR1) comprising SEQ ID NO: 38,
(ii) a heavy chain CDR2 (VH-CDR2) comprising SEQ ID NO: 39,
(iii) a heavy chain CDR3 (VH-CDR3) comprising SEQ ID NO: 40,
(iv) a light chain CDR1 (VL-CDR1) comprising SEQ ID NO: 41,
(v) a light chain CDR2 (VL-CDR2) comprising SEQ ID NO: 42, and
(vi) a light chain CDR3 (VL-CDR3) comprising SEQ ID NO: 43.

In one embodiment, the anti-FSHR antibody or antigen-binding fragment thereof comprises:

(i)
a heavy chain comprising the amino acid
sequence:
(SEQ ID NO: 44)
QVNLKESGPGILQPSQTLNLTCSFSGFSLSTSGMGVGWIRQPSGK
GLDWLAHIWWDDDKRYNPALKSRLTISKDASSNQVFLKIASVVTA
DTATYYCVQINYGNYRFDNWGHGTTLTVSSAKTTAPSVYPLAPVC
GDTTGSSVTLGCLVKGYFPEPVTLTWNSGSLSSGVHTFPAVLQSD
LYTLSSSVTVTSSTWPSQSITCNVAHPASSTKVDKKIEPRGPTIK
PCPPCKCPAPNLLGGPSVFIFPPKIKDVLMISLSPIVTCVVVDVS
EDDPDVQISWFVNNVEVHTAQTQTHREDYNSTLRVVSALPIQHQD
WMSGKEFKCKVNNKDLPAPIERTISKPKGSVRAPQVYVLPPPEEE
MTKKQVTLTCMVTDFMPEDIYVEWTNNGKTELNYKNTEPVLDSDG
SYFMYSKLRVEKKNWVERNSYSCSVVHEGLHNHHTTKSFSRTPGK,
and
(ii)
a light chain comprising the amino acid
sequence:
(SEQ ID NO: 45)
DIVLTQSPASLAVSLGQRATISCRASESVDNYGISFLNWFQQKPG
QPPKLLIYAASNQRSGVPARFSGSGSGTDFSLNIHPMEEDDTAMY
FCQQSKEVPWTFGGGTKVEIKRADAAPTVSIFPPSSEQLTSGGAS
VVCFLNNFYPKDINVKWKIDGSERQNGVLNSWTDQDSKDSIYSMS
STLTLTKDEYERHNSYTCEATHKTSTSPIVKSFNRNEC

In one embodiment, the anti-FSHR antibody or antigen-binding fragment thereof comprises:

(i)
a heavy chain comprising the amino acid
sequence:
(SEQ ID NO: 46)
EVQLVESGGGLVQPGGSLRLSCSFSGFSLSTSGMGVGWIRQAPGK
GLEWVAHIWWDDDKRYNPALKSRFTLSVDRSKNTLYLQMNSLRAE
DTATYYCVQINYGNYRFDNWGHGTLVTVSSASTKGPSVFPLAPSS
KSTSGGTAALGCLVKDYFPEPVTVSWNSGALTSGVHTFPAVLQSS
GLYSLSSVVTVPSSSLGTQTYICNVNHKPSNTKVDKKVEPKSCDK
THTCPPCPAPELLGGPSVFLFPPKPKDTLMISRTPEVTCVVVDVS
HEDPEVKFNWYVDGVEVHNAKTKPREEQYNSTYRVVSVLTVLHQD
WLNGKEYKCKVSNKALPAPIEKTISKAKGQPREPQVYTLPPSRDE
LTKNQVSLTCLVKGFYPSDIAVEWESNGQPENNYKTTPPVLDSDG
SFFLYSKLTVDKSRWQQGNVFSCSVMHEALHNHYTQKSLSLSPGK,
and
(ii)
a light chain comprising the amino acid
sequence:
(SEQ ID NO: 47)
DIQMTQSPSSLSASVGDRVTISCRASESVDNYGISFLNWFQQKPG
KAPKLLIYAASNQRSGVPSRFSGSGSGTDFTLTISSLOPEDFATY
FCQQSKEVPWTFGQGTKVEIKRTVAAPSVFIFPPSDEQLKSGTAS
VVCLLNNFYPREAKVQWKVDNALQSGNSQESVTEQDSKDSTYSLS
STLTLSKADYEKHKVYACEVTHQGLSSPVTKSFNRGEC.

In one embodiment, the anti-FSHR antibody or antigen-binding fragment thereof comprises:

(i)
a heavy chain comprising the amino acid
sequence:
(SEQ ID NO: 48)
EVQLKESGPGILQPSQTLNLTCSFSGFSLSTSGMGVGWIRQPSGK
GLDWLAHIWWDDDKRYNPALKSRLTISKDASSNQVFLKIASVVTA
DTATYYCVQINYGNYRFDNWGHGTTLTVSSASTKGPSVFPLAPSS
KSTSGGTAALGCLVKDYFPEPVTVSWNSGALTSGVHTFPAVLQSS
GLYSLSSVVTVPSSSLGTQTYICNVNHKPSNTKVDKKVEPKSCDK
THTCPPCPAPELLGGPSVFLFPPKPKDTLMISRTPEVTCVVVDVS
HEDPEVKFNWYVDGVEVHNAKTKPREEQYNSTYRVVSVLTVLHQD
WLNGKEYKCKVSNKALPAPIEKTISKAKGQPREPQVYTLPPSRDE
LTKNQVSLTCLVKGFYPSDIAVEWESNGQPENNYKTTPPVLDSDG
SFFLYSKLTVDKSRWQQGNVFSCSVMHEALHNHYTQKSLSLSPGK,
and
(ii)
a light chain comprising the amino acid
sequence:
(SEQ ID NO: 49)
DIQMTQSPASLAVSLGQRATISCRASESVDNYGISFLNWFQQKPG
QPPKLLIYAASNQRSGVPARFSGSGSGTDFSLNIHPMEEDDTAMY
FCQQSKEVPWTFGGGTKVEIKRTVAAPSVFIFPPSDEQLKSGTAS
VVCLLNNFYPREAKVQWKVDNALQSGNSQESVTEQDSKDSTYSLS
STLTLSKADYEKHKVYACEVTHQGLSSPVTKSFNRGEC

In one embodiment, the anti-FSHR antibody or antigen-binding fragment thereof comprises:

(i) a heavy chain comprising the amino acid
sequence:
(SEQ ID NO: 48)
EVQLKESGPGILQPSQTLNLTCSFSGFSLSTSGMGVGWIRQPSGK
GLDWLAHIWWDDDKRYNPALKSRLTISKDASSNQVFLKIASVVTA
DTATYYCVQINYGNYRFDNWGHGTTLTVSSASTKGPSVFPLAPSS
KSTSGGTAALGCLVKDYFPEPVTVSWNSGALTSGVHTFPAVLQSS
GLYSLSSVVTVPSSSLGTQTYICNVNHKPSNTKVDKKVEPKSCDK
THTCPPCPAPELLGGPSVFLFPPKPKDTLMISRTPEVTCVVVDVS
HEDPEVKFNWYVDGVEVHNAKTKPREEQYNSTYRVVSVLTVLHQD
WLNGKEYKCKVSNKALPAPIEKTISKAKGQPREPQVYTLPPSRDE
LTKNQVSLTCLVKGFYPSDIAVEWESNGQPENNYKTTPPVLDSDG
SFFLYSKLTVDKSRWQQGNVFSCSVMHEALHNHYTQKSLSLSPGK,
and
(ii) a light chain comprising the amino acid
sequence:
(SEQ ID NO: 47)
DIQMTQSPSSLSASVGDRVTISCRASESVDNYGISFLNWFQQKPG
KAPKLLIYAASNQRSGVPSRFSGSGSGTDFTLTISSLQPEDFATY
FCQQSKEVPWTFGQGTKVEIKRTVAAPSVFIFPPSDEQLKSGTAS
VVCLLNNFYPREAKVQWKVDNALQSGNSQESVTEQDSKDSTYSLS
STLTLSKADYEKHKVYACEVTHQGLSSPVTKSFNRGEC.

In one embodiment, the anti-FSHR antibody or antigen-binding fragment thereof comprises:

(i)
a heavy chain comprising the amino acid
sequence:
(SEQ ID NO: 46)
EVQLVESGGGLVQPGGSLRLSCSFSGFSLSTSGMGVGWIRQAPGK
GLEWVAHIWWDDDKRYNPALKSRFTLSVDRSKNTLYLQMNSLRAE
DTATYYCVQINYGNYRFDNWGHGTLVTVSSASTKGPSVFPLAPSS
KSTSGGTAALGCLVKDYFPEPVTVSWNSGALTSGVHTFPAVLQSS
GLYSLSSVVTVPSSSLGTQTYICNVNHKPSNTKVDKKVEPKSCDK
THTCPPCPAPELLGGPSVFLFPPKPKDTLMISRTPEVTCVVVDVS
HEDPEVKFNWYVDGVEVHNAKTKPREEQYNSTYRVVSVLTVLHQD
WLNGKEYKCKVSNKALPAPIEKTISKAKGQPREPQVYTLPPSRDE
LTKNQVSLTCLVKGFYPSDIAVEWESNGQPENNYKTTPPVLDSDG
SFFLYSKLTVDKSRWQQGNVFSCSVMHEALHNHYTQKSLSLSPGK,
and
(ii) a light chain comprising the amino
acid sequence:
(SEQ ID NO: 49)
DIQMTQSPASLAVSLGQRATISCRASESVDNYGISFLNWFQQKPG
QPPKLLIYAASNQRSGVPARFSGSGSGTDFSLNIHPMEEDDTAMY
FCQQSKEVPWTFGGGTKVEIKRTVAAPSVFIFPPSDEQLKSGTAS
VVCLLNNFYPREAKVQWKVDNALQSGNSQESVTEQDSKDSTYSLS
STLTLSKADYEKHKVYACEVTHQGLSSPVTKSFNRGEC.

In one embodiment, a FSHR ligand can be labeled with a gas-filled microbubbles, for use in contrast enhanced ultrasound imaging. For instance, the FSHR ligand can be associated to or incorporated in a stabilizing envelope of said gas-filled microbubbles according to conventional methods, for instance by covalently binding the ligand to an amphiphilic component, such as a phospholipid.

In one embodiment, the FSHR ligand is fluorescently labeled. In one embodiment the FSHR ligand is coupled to gold particles.

In a preferred embodiment, the FSHR ligand is radioactively labeled. In a preferred embodiment the FSHR ligand is radioactively labeled FSH or a radioactively labeled anti-FSHR antibody or antigen-binding fragment thereof.

### BRIEF DESCRIPTION OF THE DRAWINGS

FIG. 1A-M is an electrophoresis file run summary of 10 atherosclerotic plaque tissue samples and quantification of FSHR-mRNA copies present in 50 ng of total RNA. The electrophoresis profile (FIG. 1A) indicated that only four atherosclerotic plaque tissue samples (patients 4, 5, 9, and 10) and normal prostate sample had the required quality of total RNA for FSHR-mRNA quantification (FIG. 1B-M) by Nanostring methodology (i.e., visible bands for the fractions 28 s (4,000 nucleotides) and 18 s (2,000 nucleotides).

FIG. 2 is a low-power photograph of advanced plaque lesions of atherosclerotic human carotid. Scale bar: 1 mm.

FIG. 3 is a photograph of the angiogenesis associated with atherosclerotic plaque tissue in a patient (patient 198). This picture indicates that the neovessel formation occurred mainly in zones with inflammatory infiltrates. The highest microvessel density was noticed at the basal part of the intima of carotid wall (asterisks). Neovessels originating from the arterial endothelial cells are indicated by an arrowhead. This result indicates that the majority of the neovessels in the atherosclerotic plaques originated from adventitial vasa vasorum. Arrows point to the new CD34-positive blood vessels associated with the atherosclerotic plaque. Scale bar: 500 μm.

FIG. 4A-E is a combination of photographs of a double immunofluorescence labeling on atherosclerotic plaque tissue confirming the identity of endothelial cells expressing FSHR. FIG. 4A-D. An antibody against the vascular endothelial cell marker von Willebrand factor (vWF) (FIG. 4B), followed by a green-labeled secondary antibody, overlapped with the signal from the anti-FSHR323 antibody detected by a secondary red-labeled antibody (FIG. 4A). Sections were also incubated with DAPI (FIG. 4C), a marker of cell nuclei (blue color). Bar: 10 μm. FIG. 4E. vWF-positive endothelial cells (green color) of normal human mammaria used as a control are FSHR-negative. Scale bar: 10 μm.

FIG. 5A-B is a combination of photographs showing that lymphatic neovessels associated with atherosclerotic plaques did not express FSHR. Immunohistochemical analysis was performed on paraffin-embedded sections of human atherosclerotic tissues with the use of the anti-FSHR monoclonal antibody 323, followed by a secondary peroxidase-coupled antibody visualized with the use of the brown peroxidase reaction product. Sections were also stained with hematoxylin. In serial sections of an atherosclerotic plaque tissue, the blood neovessels showed a strong presence of FSHR (FIG. 5A, arrows) whereas the lymphatic vessels (asterisks), identified with the use of the monoclonal antibody D2-40 (FIG. 5B), did not express FSHR. Scale bars: 50 μm.

FIG. 6 is a photograph showing FSHR and alpha-SMC actin colocalization in blood neovessels associated with atherosclerotic plaques in late stages of disease. Colocalization of vascular endothelial FSHR (brown color) and CD31 (red color), a marker of endothelial cells, indicated that FSHR is expressed in both the mature neovessels (ex., small arterioles in FIG. 8A) and the immature blood neovessels (ex., ruptured venule in FIG. 7B). Scale bar: 25 μm.

FIG. 7A-B is a combination of photographs showing FSHR colocalization with the endothelial marker CD31 in blood neovessels associated with atherosclerotic plaques in late stages of disease. Colocalization of vascular endothelial FSHR (brown color) and CD31 (red color), a marker of endothelial cells, in both mature neovessels (ex., small arterioles in FIG. 7A) and immature blood neovessels (ex., ruptured venule (in FIG. 7B). Scale bars: 50 μm.

FIG. 8A-B is a combination of photographs showing expression of FSHR by macrophages in human atherosclerotic plaques. Immunohistochemical analysis was performed on paraffin-embedded sections of human atherosclerotic tissues with the use of (i) the anti-FSHR monoclonal antibody 323, followed by a secondary peroxidase-coupled antibody visualized with the use of the insoluble brown peroxidase reaction product, and (ii) the antibody against the M2-macrophage marker CD163, followed by an alkaline phosphatase-coupled secondary antibody visualized with the use of the red alkaline phosphatase-reaction product. The same sections were also stained with hematoxylin (blue nuclei). FIG. 8A strong signal for FSHR was detected on the cell surface membranes (arrows in FIG. 8A, arrowheads in FIG. 8B) and in the cytoplasm of M1-macrophages and M1-macrophage-derived foam cells (asterisks in FIG. 8B). By contrast, no colocalization of FSHR and CD163 was detected in M2-macrophages and in M2-macrophage-derived foam cells (arrows in FIG. 8B). Scale bars: 50 μm.

FIG. 9A-C is a combination of photographs showing staining for FSHR on multinucleated giant cells associated with atherosclerotic plaques. Standard peroxidase immunohistochemistry with the use of mouse anti-hFSHR monoclonal antibody 323 (FIG. 9A), an irrelevant IgG2a antibody (FIG. 9B), and CD34, a marker of endothelial cells (FIG. 9C). Multinucleated giant cells (asterisks), which are CD34-negative, express FSHR mainly on their cell surface membrane. No signal was noticed for the control antibody (irrelevant mouse IgG2a antibody M9144 from Sigma). Scale bars: 50 μm.

FIG. 10 is a photograph showing that leukocytes present in the atherosclerotic plaque tissues did not express FSHR. By contrast, a strong brown staining for FSHR was observed in blood microvessels. Scale bar: 50 μm.

FIG. 11 is a photograph showing expression of hFSHR in the atherosclerotic plaques of ApoE-KO/hFSHR-KI mice fed for five months with an atherogenic diet rich in cholesterol. The immunohistochemical analysis was performed on paraffin-embedded sections of aortic tissues with the use of the biotinylated anti-FSHR monoclonal antibody 323, followed by streptavidin-peroxidase conjugate visualized with the use of the insoluble brown peroxidase reaction product. The same sections were also stained with hematoxylin (blue nuclei). A strong signal for FSHR was detected on the arterial endothelium and smooth muscle cells. Scale bar: 100 μm. Inset: Fat cells (FC) derived from either macrophages (asterisks) or smooth muscle cells (arrowheads) were also FSHR-positive. Scale bar: 50 μm.

FIG. 12A-E is a combination of photographs showing localization of FSHR323-colloidal gold particles in the atherosclerotic plaques in ApoE-KO/hFSHR-KI mice fed for 5 months with an atherogenic diet containing 0.5% cholesterol. After 20 min of circulation in vivo the tracer particles labeled the luminal plasma membrane and clathrin-coated pits (FIG. 12A), clathrin-coated vesicles (FIG. 12B), and endosomes (FIG. 12C) of aortic endothelial cells covering the atherosclerotic plaques. A large amount of free gold labeled FSHR323 was noticed in the interstitial tissue of the atherosclerotic plaques (FIG. 12D) and some tracer was already internalized via clathrin-coated pits and clathrin-coated vesicles into macrophages associated with the atherosclerotic plaques (FIG. 12E). Scale bars: 100 nm.

TABLE OF SEQUENCES

| SEQ ID | Sequence | |
|---|---|---|
| NO | function | Sequence |
| 1 | VH-CDR1 | QFYVG |
| 2 | VH-CDR1 | RQWVI |
| 3 | VH-CDR1 | KQWLL |
| 4 | VH-CDR1 | RSWIL |
| 5 | VH-CDR1 | KYWTQ |
| 6 | VH-CDR2 | EIFPRTGNTNYNEKFKG |
| 7 | VH-CDR2 | EILPRNGNTNYNEKFKG |
| 8 | VH-CDR2 | EIFPRNGNTNYNEKFKG |
| 9 | VH-CDR2 | EIYPQNQNTNYNEKFKG |
| 10 | VH-CDR2 | EIYPRNGNTNYNEKFKG |
| 11 | VH-CDR3 | GPTASGYAMDY |
| 12 | VL-CDR1 | RSSQSIVHRNGNTYLE |
| 13 | VL-CDR2 | KVSNRFS |
| 14 | VL-CDR3 | FQGSHVPFT |
| 15 | VH chain | QVQLQQSGAELMKPGASVKISCKATGYTFSQFYVGWV |
| | | KQRPGHGLEWIGEIFPRTGNTNYNEKFKGKATFTADTSS |
| | | STAYMQLSSLTSEDSAVYYCARGPTASGYAMDYWGQG |
| | | TSVTVSS |
| 16 | VH chain | QVQLQQSGAELMKPGASVKISCKATGYTFSRQWVIWVK |
| | | QRPGHGLEWIGEILPRNGNTNYNEKFKGKATFTADTSSS |
| | | TAYMQLSSLTSEDSAVYYCARGPTASGYAMDYWGQGT |
| | | SVTVSS |
| 17 | VH chain | QVQLQQSGAELMKPGASVKISCKATGYTFSKQWLLWV |
| | | KQRPGHGLEWIGEIFPRNGNTNYNEKFKGKATFTADTSS |
| | | STAYMQLSSLTSEDSAVYYCARGPTASGYAMDYWGQG |
| | | TSVTVSS |
| 18 | VH chain | QVQLQQSGAELMKPGASVKISCKATGYTFSRSWILWVK |
| | | QRPGHGLEWIGEIYPQNQNTNYNEKFKGKATFTADTSS |
| | | STAYMQLSSLTSEDSAVYYCARGPTASGYAMDYWGQGT |
| | | SVTVSS |
| 19 | VH chain | QVQLQQSGAELMKPGASVKISCKATGYTFSKYWTQWV |
| | | KQRPGHGLEWIGEIYPRNGNTNYNEKFKGKATFTADTSS |
| | | STAYMQLSSLTSEDSAVYYCARGPTASGYAMDYWGQG |
| | | TSVTVSS |
| 20 | VL chain | DVLMTQTPLSLPVSLGDQASISCRSSQSIVHRNGNTYLE |
| | | WYLQKPGQSPKLLIYKVSNRFSGVPDRFSGSGSGTDFTL |
| | | KIIRVEAEDLGVYYCFQGSHVPFTFGSGTKLEIK |
| 21 | Primer APOE1 | GCCTAGCCGAGGGAGAGCCG |
| 22 | Primer APOE2 | TGTGACTTGGGAGCTCTGCAGC |
| 23 | Primer APOE3 | GCCGCCCCGACTGCATCT |
| 24 | Primer FSHRWTF | AAGCATCCTTTAGTGGGTCA |
| 25 | Primer FSHRKIF | CCTTACATGTTTTACTAGCCAG |
| 26 | Primer FSHRR | GCCAGTGATGACATCCAGAT |
| 27 | Human follicle- | MDYYRKYAAIFLVTLSVFLHVLHSAPDVQDCPECTLQE |
| | stimulating | NPFFSQPGAPILQCMGCCFSRAYPTPLRSKKTMLVQKNV |
| | hormone | TSESTCCVAKSYNRVTVMGGFKVENHTACHCSTCYYH |
| | alpha chain | KS |
| 28 | Human follicle- | MKTLQFFFLFCCWKAICCNSCELTNITIAIEKEECRFCISI |
| | stimulating | NTTWCAGYCYTRDLVYKDPARPKIQKTCTFKELVYETV |
| | hormone | RVPGCAHHADSLYTYPVATQCHCGKCDSDSTDCTVRGL |
| | beta subunit | GPSYCSFGEMKE |
| 29 | Human follicle- | HHHHHHNSCELTNITIAIEKEECRFCISINTTWCAGYCYT |
| | stimulating | RDLVYKDPARPKIQKTCTFKELVYETVRVPGCAHHADS |
| | hormone | LYTYPVATQCHCGKCDSDSTDCTVRGLGPSYCSFGEMK |
| | beta subunit | EKDEL |
| 30 | VH-CDR1 | SDYAWN |
| 31 | VH-CDR2 | SIFSSGSINYNPSLKS |
| 32 | VH-CDR3 | GGTGTDY |
| 33 | VL-CDR1 | RASQDISNYLS |
| 34 | VL-CDR2 | YTSRLHS |
| 35 | VL-CDR3 | QQGHTLPPT |
| 36 | VH chain | DVHLQESGPGLVKPSQSLSLTCTVTGFSITSDYAWNWIR |
| | | QFPGNKLEWMGSIFSSGSINYNPSLKSRISITRDTSRNQFF |
| | | LQLNSVTTADAGTYYCARGGTGTDYWGQGTTLTVSS |
| 37 | VL chain | DIQMTQTTSSLSASLGDRVTISCRASQDISNYLSWYQQK |
| | | PDGTIKLLIYYTSRLHSGVSSRFSGSGSGTDYSLTISNLEQ |
| | | EDFATYFCQQGHTLPPTFGGGTKLEIK |
| 38 | VH-CDR1 | TSGMGVG |
| 39 | VH-CDR2 | HIWWDDDKRYNPALKS |
| 40 | VH-CDR3 | INYGNYRFDN |
| 41 | VL-CDR1 | RASESVDNYGISFLN |
| 42 | VL-CDR2 | AASNQRS |
| 43 | VL-CDR3 | QQSKEVPWT |
| 44 | VH chain | QVNLKESGPGILQPSQTLNLTCSFSGFSLSTSGMGVGWIR |
| | | QPSGKGLDWLAHIWWDDDKRYNPALKSRLTISKDASSN |
| | | QVFLKIASVVTADTATYYCVQINYGNYRFDNWGHGTTL |
| | | TVSSAKTTAPSVYPLAPVCGDTTGSSVTLGCLVKGYFPE |
| | | PVTLTWNSGSLSSGVHTFPAVLQSDLYTLSSSVTVTSST |
| | | WPSQSITCNVAHPASSTKVDKKIEPRGPTIKPCPPCKCPA |
| | | PNLLGGPSVFIFPPKIKDVLMISLSPIVTCVVVDVSEDDPD |
| | | VQISWFVNNVEVHTAQTQTHREDYNSTLRVVSALPIQH |
| | | QDWMSGKEFKCKVNNKDLPAPIERTISKPKGSVRAPQV |
| | | YVLPPPEEEMTKKQVTLTCMVTDFMPEDIYVEWTNNGK |
| | | TELNYKNTEPVLDSDGSYFMYSKLRVEKKNWVERNSYS |
| | | CSVVHEGLHNHHTTKSFSRTPGK |
| 45 | VL chain | DIVLTQSPASLAVSLGQRATISCRASESVDNYGISFLNWF |
| | | QQKPGQPPKLLIYAASNQRSGVPARFSGSGSGTDFSLNIH |
| | | PMEEDDTAMYFCQQSKEVPWTFGGGTKVEIKRADAAPT |
| | | VSIFPPSSEQLTSGGASVVCFLNNFYPKDINVKWKIDGSE |
| | | RQNGVLNSWTDQDSKDSIYSMSSTLTLTKDEYERHNSY |
| | | TCEATHKTSTSPIVKSFNRNEC |
| 46 | VH chain | EVQLVESGGGLVQPGGSLRLSCSFSGFSLSTSGMGVGWI |
| | | RQAPGKGLEWVAHIWWDDDKRYNPALKSRFTLSVDRS |
| | | KNTLYLQMNSLRAEDTATYYCVQINYGNYRFDNWGHG |
| | | TLVTVSSASTKGPSVFPLAPSSKSTSGGTAALGCLVKDY |
| | | FPEPVTVSWNSGALTSGVHTFPAVLQSSGLYSLSSVVTV |
| | | PSSSLGTQTYICNVNHKPSNTKVDKKVEPKSCDKTHTCP |
| | | PCPAPELLGGPSVFLFPPKPKDTLMISRTPEVTCVVVDVS |
| | | HEDPEVKFNWYVDGVEVHNAKTKPREEQYNSTYRVVS |
| | | VLTVLHQDWLNGKEYKCKVSNKALPAPIEKTISKAKGQ |
| | | PREPQVYTLPPSRDELTKNQVSLTCLVKGFYPSDIAVEW |
| | | ESNGQPENNYKTTPPVLDSDGSFFLYSKLTVDKSRWQQ |
| | | GNVFSCSVMHEALHNHYTQKSLSLSPGK |
| 47 | VL chain | DIQMTQSPSSLSASVGDRVTISCRASESVDNYGISFLNWF |
| | | QQKPGKAPKLLIYAASNQRSGVPSRFSGSGSGTDFTLTIS |
| | | SLQPEDFATYFCQQSKEVPWTFGQGTKVEIKRTVAAPSV |
| | | FIFPPSDEQLKSGTASVVCLLNNFYPREAKVQWKVDNAL |
| | | QSGNSQESVTEQDSKDSTYSLSSTLTLSKADYEKHKVYA |
| | | CEVTHQGLSSPVTKSFNRGEC |
| 48 | VH chain | EVQLKESGPGILQPSQTLNLTCSFSGFSLSTSGMGVGWIR |
| | | QPSGKGLDWLAHIWWDDDKRYNPALKSRLTISKDASSN |
| | | QVFLKIASVVTADTATYYCVQINYGNYRFDNWGHGTTL |
| | | TVSSASTKGPSVFPLAPSSKSTSGGTAALGCLVKDYFPEP |
| | | VTVSWNSGALTSGVHTFPAVLQSSGLYSLSSVVTVPSSS |
| | | LGTQTYICNVNHKPSNTKVDKKVEPKSCDKTHTCPPCPA |
| | | PELLGGPSVFLFPPKPKDTLMISRTPEVTCVVVDVSHEDP |
| | | EVKFNWYVDGVEVHNAKTKPREEQYNSTYRVVSVLTV |
| | | LHQDWLNGKEYKCKVSNKALPAPIEKTISKAKGQPREP |
| | | QVYTLPPSRDELTKNQVSLTCLVKGFYPSDIAVEWESNG |
| | | QPENNYKTTPPVLDSDGSFFLYSKLTVDKSRWQQGNVF |
| | | SCSVMHEALHNHYTQKSLSLSPGK |
| 49 | VL chain | DIQMTQSPASLAVSLGQRATISCRASESVDNYGISFLNWF |
| | | QQKPGQPPKLLIYAASNQRSGVPARFSGSGSGTDFSLNIH |
| | | PMEEDDTAMYFCQQSKEVPWTFGGGTKVEIKRTVAAPS |
| | | VFIFPPSDEQLKSGTASVVCLLNNFYPREAKVQWKVDN |
| | | ALQSGNSQESVTEQDSKDSTYSLSSTLTLSKADYEKHKV |
| | | YACEVTHQGLSSPVTKSFNRGEC |

### EXAMPLES

The present invention is further illustrated by the following example.

### Example 1

### Materials and Methods

### Human Tissue Samples

Paraffin sections for atherosclerotic plaques (50 patients) and normal tissues (9 donors) were used for the experiments.

### Immunohistochemistry

Paraffin sections (5 μm) of human atherosclerotic lesions were immunolabeled with antibodies directed against the endothelial markers CD34, von Willebrand Factor and FSHR, the T-cell markers CD3 and CD8, M-1 macrophage marker CD68, and M-2 macrophage marker CD163 (Table 1). An irrelevant mouse IgG2a monoclonal antibody of the same isotype as FSHR-323 was used as control. Immunohistochemistry was carried out using an automated immunohistochemical stainer according to the manufacturer's guidelines (Leica Bond RX, Leica Biosystems). Antigen retrieval was conducted by treatment with high temperature at pH 6 or pH 9 (Table 1). Afterwards, slides were manually washed using hot water supplemented with detergent, followed by tap water only and distilled H2O in the final step. For dehydration, the slides were transferred to an ascending ethanol series (2×80%, 2×96%, 2×abs. EtOH; 3 min each). After dehydration, the slides were transferred to xylene (3×2 min) and automatically embedded in Pertex.

### Indirect Immunofluorescence Confocal Microscopy

Paraffin sections (5 μm) of human atherosclerotic lesions were immunolabeled with antibodies directed against the endothelial marker von Willebrand Factor and FSHR. To block the nonspecific binding of antibodies the slides were incubated 1 hour at room temperature with 2% goat serum in PBS (GS-PBS). Double labelling experiments have been done with atherosclerotic plaque tissue sections incubated with a mixture of FSHR323 antibody (dilution 0.2 μg/ml) in GS-PBS) and the rabbit polyclonal anti-von Willebrand factor, a specific marker of endothelial cells (Sigma; dilution 1:3 000). Mature blood vessels were determined by using a mixture of mouse anti-human alpha-SMC actin monoclonal antibody and the rabbit polyclonal anti-von Willebrand factor. A mixture of goat-anti mouse IgG-Alexa 555 and goat-anti rabbit Ig-Alexa 488 (Molecular Probes; dilution 1:750) has been used as secondary antibodies. The cell nuclei were detected by incubating slides for 10 min with DAPI (Molecular Probes; dilution 1:1000 in PBS). The slides were mounted in Dako® fluorescent mounting medium containing 15 mM sodium azide and examined with a Zeiss 510 Confocal Laser Scanning Microscope. Negative controls consisted of umbilical cord tissues, human mammary artery, and human normal coronary artery.

### Histopathological Evaluation

The histopathological evaluation was performed by a pathologist of Institut Curie. For the evaluation of anti-FSH-R staining of atherosclerotic plaque cells an intensity score (IS) specifying negative (0), weak (1+), moderate (2+) or strong (3+) staining was used. Within the atherosclerotic plaque area a distinction was made between a homogeneous and a heterogeneous staining pattern. A homogeneous staining pattern thereby implied a constant staining intensity (weak, moderate or strong) of plaque cells, whereas a heterogeneous staining pattern implied varying staining intensities of plaque cells within the same plaque area. Furthermore, the predominantly stained subcellular compartment (m: membrane, n: nucleus, c: cytoplasm) of cells was determined.

### Direct and Multiplexed FSHR-RNA Quantification

Total RNA was extracted from paraffin embedded human atherosclerotic plaque tissues (10 patients) according to Qiagen miRNEasy protocol (Cat No: 217004, Courtaboeuf, France). Total RNA concentration and purity (ratio 260/280 and ratio 260/230 nm) were measured using a Nanodrop ND8000 spectrophotometer (Ozyme, Saint-Quentin en Yvelines, France). Total RNA integrity was assessed by micro electrophoresis (RNA6000 LabChip, Agilent technologies, Les Ulis, France), and RNA Integrity Number was calculated, upon a total RNA migration. Direct quantification of mRNA was achieved according to a Nanostring Custom Elements approach (Nanostring, Seattle, USA). Briefly, total RNA (50 ng) was used as template to detect 94 targets corresponding to 86 mRNA of interest, and 8 housekeeping genes. The Nanostring Element chemistry was chosen for its flexibility (http://www.nanostring.com/elements/tagsets). This approach required intermediate oligonucleotides (a probe A and probe B) for each target designed by Nanostring. The long oligonucleotides were produced by Integrated DNA Technologies (IDT; Leuven, Belgium). Secondary oligonucleotides, complementary to 5′ tail of first IDT long oligonucleotides, were biotinylated or coupled to a reporter tag specific to each target. Tags set kits were ordered from Nanostring (Seattle, USA). A universal human prostate RNA and water were also hybridized in parallel. Positive and negative controls were also added to samples as spikes in controls. The Nanostring nSolver software was used to control raw data and to normalize data based on geometric means of positive controls. Water was used to deduct unspecific counts. Analysis of variance (Anova) was performed between normal carotid tissue (n=2 samples) and carotid atherosclerotic lesions (n=10 samples) normalized data.

### Production of a Mouse Model for Human Atherosclerosis: Transgenic ApoE-KO Mice Expressing the Human FSHR

For this study we have generated ApoE-KO mice expressing the human FSHR by breeding ApoE-KO female mice purchased from Jackson Laboratory (Chicago, USA; Ref: B6.129P2) with hFSHR-KI male mice (CHIPHE Laboratory, Marseille, France). By using a genotyping protocol (see below), double homozygous ApoE-KO-hFSHR-KI mice have been selected to induce atherosclerotic lesions with high fed cholesterol diet.

### Genotyping Protocols

Strain name: APOE

### Reagents and Solutions

Kapa Mouse Genotyping kit (Sigma Ref: KK7352)
Primers 30 μM (Eurofins)
Mineral Oil (Sigma Ref: 330779-1L)
DNA (extracted with Kapa Mouse Genotyping kit)

### Reaction Mix

| MIX | Quantity/well |
|---|---|
| Water | 6.80 μl |
| 2X Kapa Buffer | 10.00 μl |
| APOE1 | 0.60 μl |
| APOE2 | 0.30 μl |
| APOE3 | 0.30 μl |
| DNA | 2.00 μl |
| Final Quantity | 20.00 μl |

### PCR Program

| | | | |
|---|---|---|---|
| 3 | min | 95° C. | |
| 15 | sec | 94° C. | 35 cycles |
| 15 | sec | 60° C. | |
| 30 | sec | 72° C. | |
| 5 | min | 72° C. | |

Migration: Migration on 1.5% agarose gels.

### Primers Sequences

APOE1
(SEQ ID NO: 21)
5′ GCCTAGCCGAGGGAGAGCCG 3′
APOE2
(SEQ ID NO: 22)
5′ TGTGACTTGGGAGCTCTGCAGC 3′
APOE3
(SEQ ID NO: 23)
5′ GCCGCCCCGACTGCATCT 3′

### Expected Amplifications

WT allele: 155 bp
Modified allele: 245 bp
Strain name: FSHR

### Reagents and Solutions

Kapa Mouse Genotyping kit (Sigma Ref: KK7352)
Primers 30 μM (Eurofins)
Mineral Oil (Sigma Ref.: 330779-1L)
DNA (extracted with Kapa Mouse Genotyping kit)

### Reaction Mix

| MIX | Quantity/well |
|---|---|
| Water | 6.80 μl |
| 2X Kapa Buffer | 10.00 μl |
| FSHRWTF | 0.30 μl |
| FSHRKIF | 0.30 μl |
| FSHRR | 0.60 μl |
| DNA | 2.00 μl |
| Final Quantity | 20.00 μl |

### PCR Program

| | | | |
|---|---|---|---|
| 3 | min | 95° C. | |
| 15 | sec | 94° C. | 35 cycles |
| 15 | sec | 55° C. | |
| 30 | sec | 72° C. | |
| 5 | min | 72° C. | |

Migration: Migration on 1.5% agarose gels.

### Primer Sequences

FSHRWTF
(SEQ ID NO: 24)
5′ AAGCATCCTTTAGTGGGTCA 3′
FSHRKIF
(SEQ ID NO: 25)
5′ CCTTACATGTTTTACTAGCCAG 3′
FSHRR
(SEQ ID NO: 26)
5′ GCCAGTGATGACATCCAGAT 3′

### Expected Amplifications

WT allele: 206 bp
Modified allele: 265 bp

### Immunohistochemical Analysis of FSHR Protein Expression in the Atherosclerotic Plaques in Mice

The experiments were carried out on 12 double homozygous ApoE-KO-hFSHR-KI mice divided in two groups (A, B), each group consisting of 3 males and 3 females. The mice of groups A were fed with an atherogenic diet containing 0.5% cholesterol. Mice of group B were fed a standard diet for mice.

At 30-weeks of age the mice were sacrificed by cervical dislocation, and specimens of the aortic arch, heart, lung, pancreas, kidney, and testis were collected, fixed in 4% formaldehyde in PBS buffer, pH 7.4 (24 h, 4° C.), dehydrated in ethylic alcohol, and finally embedded in paraffin. The expression of FSHR was detected with the use of mouse anti-hFSHR biotinylated 323 antibody followed by incubation with a streptavidin-peroxidase complex.

### Immunoelectron Microscopy

The experiments were carried out on 24 double homozygous ApoE-KO-hFSHR-KI mice (3 males and 3 females/group). FSHR323-Au6 nm and mouse IgG2a isotype control antibody-Au6 nm conjugates were prepared according to standard methods (DeMey, 1986). After light anesthesia of double homozygous ApoE-KO-hFSHR-KI mice with a mixture of 10% Imalgene+5% Rompun in 0.9% sodium chloride, thoracotomy, and exposure of the heart, the tracers (200 μl, A540 nm=1) were injected in the left ventricle of mice and maintained in circulation for 20 min. After 20 min the mice were sacrificed and the heart, thoracic aorta, lung, liver, pancreas, and testis of mice were fixed at 4° C. for 24 h with a mixture of 4% paraformaldehyde+2.5% glutaraldehyde in 0.1 M cacodylate buffer, pH 7.2. After 24 h from each tissue 6-10 specimens were collected and processed for electron microscopy. Gold particles present on random cell profiles of endothelial cells were allocated to one of the following organelles: cell surface membrane, coated pits and vesicles, caveolae, endothelial junctions, endosomes (smooth vesicles and tubular structures), multivesicular bodies, and lysosomes.

### Effect of High Fed Cholesterol Diet on the Total Plasma Cholesterol in Homozygous ApoE-KO-hFSHR-KI Mice

At age of 30 weeks 200 μl of blood/mouse was harvested and plasma has been separated from blood cells by centrifugation at 4,000 g. One μl of plasma was diluted 100-fold with PBS and the total plasma cholesterol level was determined using the Abcam kit (Ref. ab65390) as indicated by supplier.

TABLE 1
Antibodies used in the present IHC study

| Antibody | Specificity | Description | Isotype | Antigen retrieval | Dilution | Incubation | Supplier |
|---|---|---|---|---|---|---|---|
| FSHR-323 | Hu-FSHR | Mouse monoclonal | IgG2a | Citrate - pH 6 | 3 μg/ml | 30 min | Institut Curie |
| F3520 | Hu-vWF | Rabbit polyclonal | — | Citrate - pH 6 | 1/3000 | 30 min | Sigma |
| M0823 (Clone JC70A) | CD31 | Mouse monoclonal | IgG1 | Tris EDTA - pH 9 | 1/100 | 30 min | Dako |
| M7165 Qbend-10 | CD34 | Mouse monoclonal | IgG1 | Tris EDTA - pH 9 | 1/200 | 30 min | Dako |
| M3619 (Clone D2-40) | Podoplanin | Mouse monoclonal | IgG1 | Citrate - pH 6 | 1/100 | 30 min | Dako |
| CD3 | CD3 | Rabbit polyclonal | — | Tris EDTA - pH 9 | 1/400 | 30 min | Dako |
| IS623 | CD8 | Mouse monoclonal | IgG1 | Tris EDTA - pH 9 | 1/100 | 30 min | Dako |
| KP1 | CD68 | Mouse monoclonal | lgG1 | Tris EDTA - pH 9 | 1/500 | 30 min | Dako |
| NCL-L CD163 (Clone 10D6) | CD163 | Mouse monoclonal | IgG1 | Tris EDTA - pH 9 | 1/500 | 30 min | Novocastra |
| M9144 | Irrelevant antigen | Mouse monoclonal | IgG2a | Citrate - pH 6 | 3 μg/ml | 30 min | Sigma |
| M0851 (Clone 1A4) | αSMC-actin | Mouse monoclonal | IgG2a | Citrate - pH 6 | 1/200 | 30 min | Dako |

### Results

The follicle-stimulating hormone receptor (FSHR) is a glycosylated transmembrane receptor that belongs to the family of G-protein coupled receptors. It binds follicle stimulating hormone (FSH) which is a central hormone of mammalian reproduction. In physiological conditions FSHR is mainly expressed in ovarian and testicular tissue (Vannier et al., 1996; Vu Hai et al., 2004) as well as in the female reproductive tract (Stilley et al., 2016). FSHR expression was observed within endothelial cells of blood vessels associated with various pathological conditions (ex., cancer (Radu et al., 2010), benign prostatic hyperplasia (Radu et al., 2010), and endometriosis (Robin et al., 2016; Ponikwicka-Tyszko et al., 2016). The endothelial FSHR seems to be linked to the angiogenesis process that also occurs in atherosclerosis, but no published data are available.

To analyze FSHR expression and localization in human atherosclerosis lesions we used immunohistochemistry and confocal microscopy on paraffin sections of 50 atherosclerotic plaque tissue specimens. Aortic arch tissue specimens from double homozygous ApoE-KO-human FSHR-KI mice fed with a diet rich in cholesterol were used to localize ultrastructurally the endothelial human FSHR in the atherosclerotic plaques developed in this animal model for human atherosclerosis.

### FSHR-mRNA Expression in Atherosclerotic Plaques

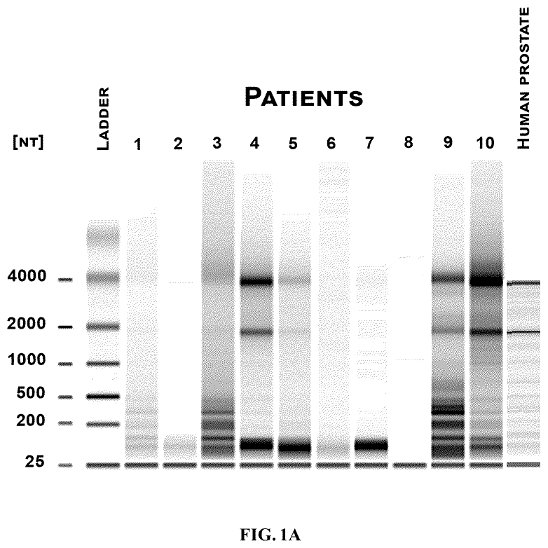

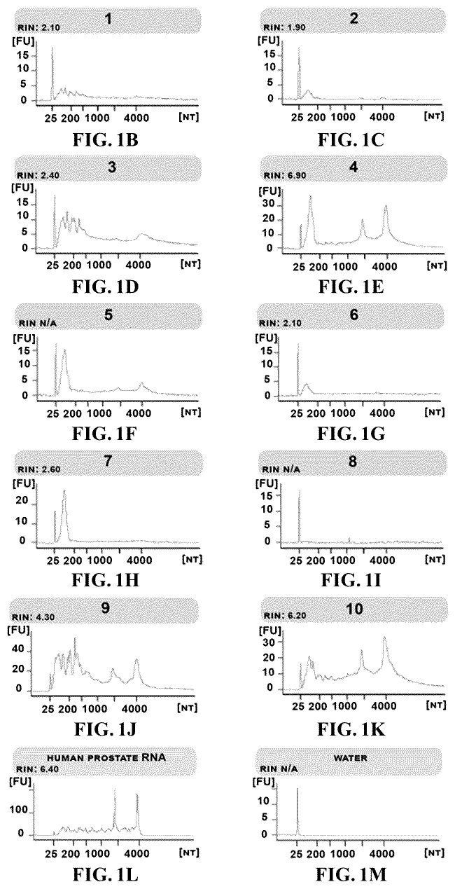

The total RNA integrity of atherosclerotic plaque tissues assessed by micro electrophoresis is shown in FIG. 1A-M. Only 4/10 samples of atherosclerotic plaque tissues had an acceptable electrophoretic profile with quantifiable bands for 28 s (4,000 nucleotides) and 18 s (2,000 nucleotides) for further RNA analysis by Nanostring technology, which detects low numbers of gene copies (Table 2). Because total RNA from human arteries was not available on the market, universal RNA from human normal prostate was used as control. The numbers of counts for FSHR-mRNA for each quantifiable sample were: 21 for sample patient 4; 33 for 5, 25 for 9, 36 for sample patient 10 and 1 for the normal human prostate tissue sample. Thus, an average of 28.8±6.9 counts of FSHR-mRNA was detected with use of the Nanostring methodology in atherosclerotic plaques associated with human carotid samples.

### Expression of FSHR-Protein in Human Atherosclerotic Plaques

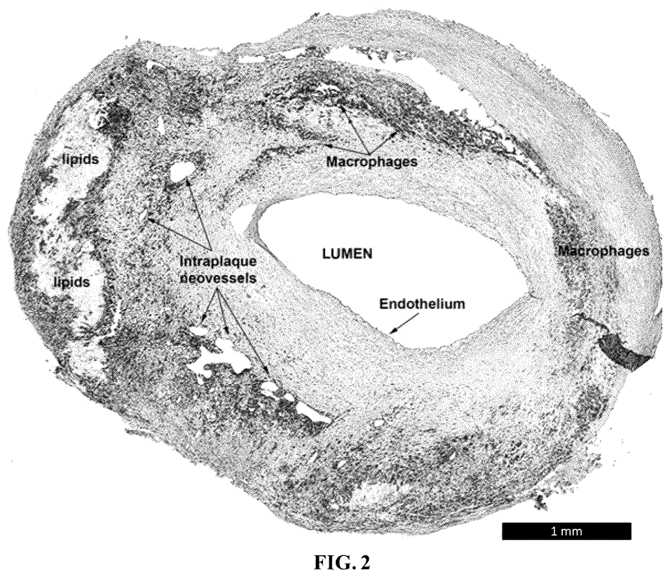

We investigated by immunohistochemistry the presence of the FSHR in advanced atherosclerotic plaques associated with carotid (44 patients), femoral artery (5 patients), and thoracic aorta (1 patient). A representative picture of an atherosclerotic plaque in human carotids is illustrated in FIG. 2.

The staining pattern for FSHR was examined on the arterial endothelium, new blood microvessels, macrophages, foam cells, and lymphocytes associated with the atherosclerotic plaques. The mouse anti-human FSHR-monoclonal antibody 323 used in this study was recently proved suitable for FSHR target validation in an IHC setting for paraffin embedded tissues (Möker et al., 2017). A representative picture of an atherosclerotic plaque in human carotids is illustrated in FIG. 2.

Neovessels Associated with Human Atherosclerotic Plaques

Published evidence indicates that plaque angiogenesis is frequently associated with inflammatory infiltrates and gradually increases with lesion progression (Sluimer and Daemen 2009).

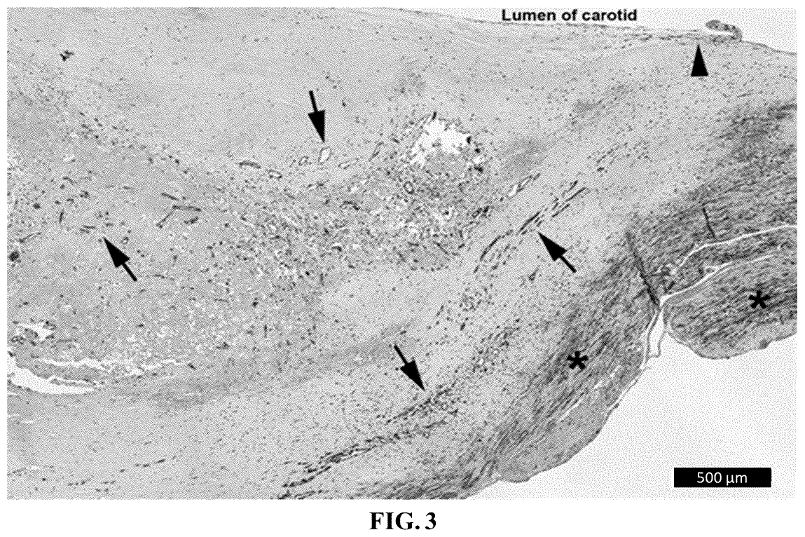

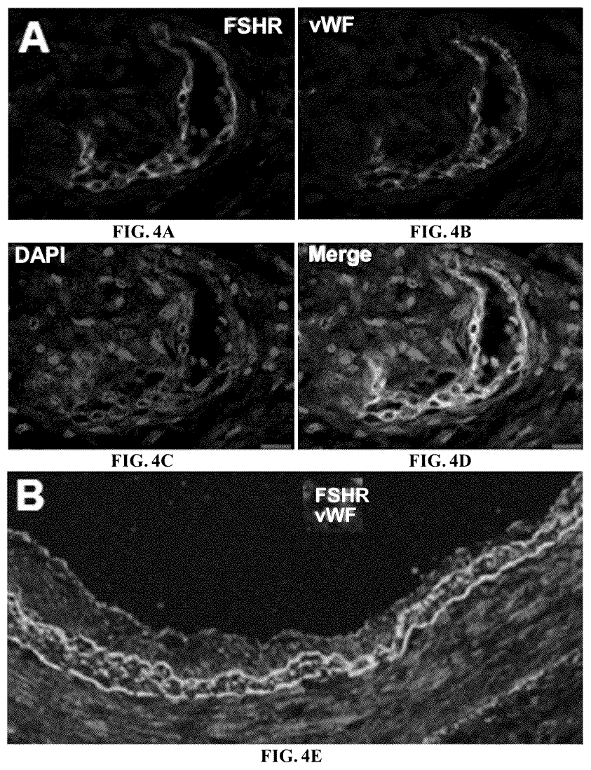

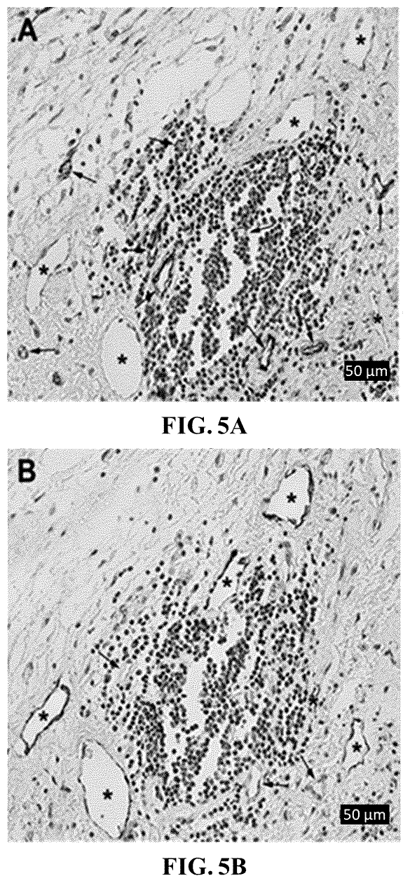

Two types of neovessels in the atherosclerotic intima are formed during the late stages of atherosclerosis: blood vessels (detected with antibodies against the endothelial markers CD31, CD34, and von Willebrand factor) and lymphatic vessels (detected with the D2-40 antibody raised against podoplanin, a marker of lymphatics). By using CD34 as an endothelial cell marker we noticed that the highest density of new blood vessels was observed on the basal front of the atherosclerotic plaques (FIG. 3), a result confirmed by other studies (Virmani et al., 2005). Double labeling for FSHR with each of these markers indicated the endothelial expression of FSHR only in arterial endothelium covering the atherosclerotic plaques and in blood neovessels nourishing the atherosclerotic plaque tissues (FIG. 4A-E). By contrast, the endothelial cells of normal mammaria artery, used as a negative control, did not express FSHR (FIG. 4E). Importantly, lymphatic neovessels when present in the atherosclerotic plaques did not express FSHR (FIG. 5A-B, asterisks).

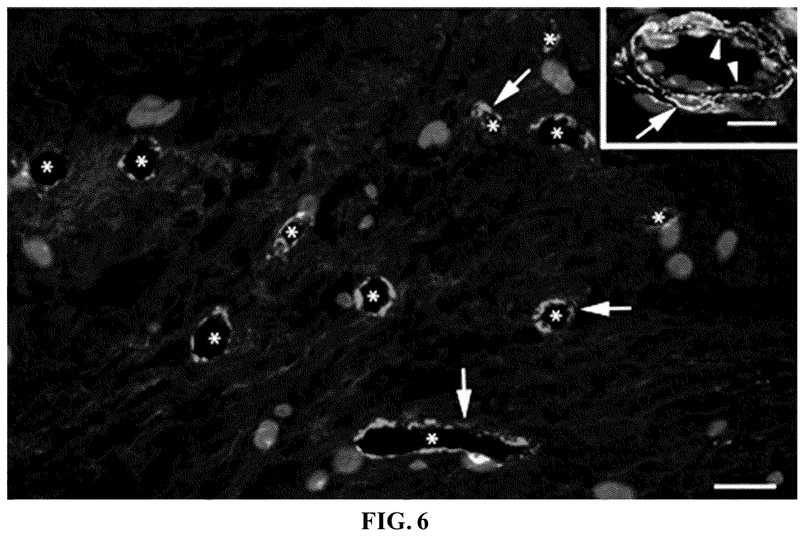

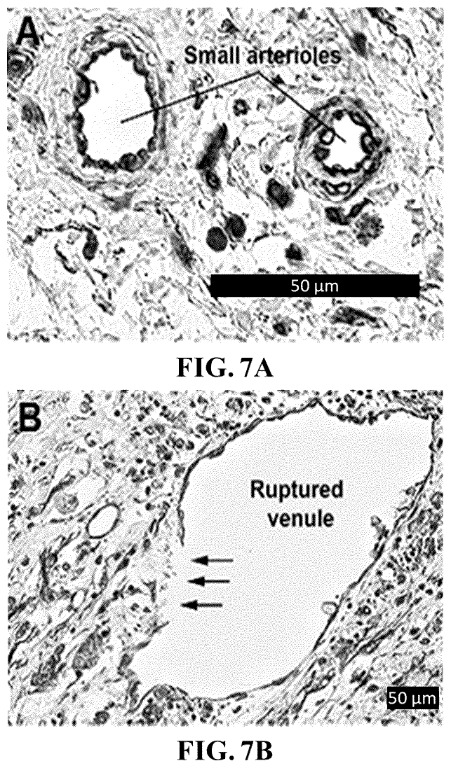

In late stages of atherosclerosis, the presence of mature and immature FSHR-positive blood vessels is a characteristic feature of atherosclerotic plaques. While the mature blood neovessels (their walls consist of alpha SMC actin-positive-vascular smooth muscle cells and pericytes) (FIG. 6, inset) predominated in stable plaques, more than 70% of blood neovessels were immature (their wall consists only of endothelial cells) and associated with unstable, rupture-prone atherosclerotic plaques (FIG. 6).

### Macrophages

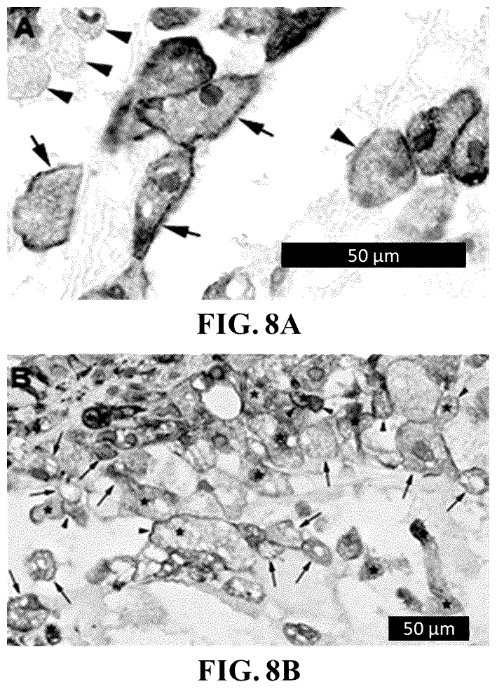

Two main types of macrophages are present in human atherosclerotic plaques: M1- and M2-macrophages (Moore & Tabas 2016). While M1-macrophages are pro-inflammatory and thus pro-atherogenic, the M2-macrophages are anti-inflammatory, and therefore, stabilize the atherosclerotic plaques. As illustrated in FIG. 8A-B, a membrane positive signal for FSHR was seen on the pro-inflammatory M1-macrophages's and foam cells derived from M1-macrophages (asterisks). No significant FSHR signal was noticed for CD163-positive M2-macrophages and M2-macrophage-derived foam cells (FIG. 8A-B, arrows).

### Multinucleated Giant Cells

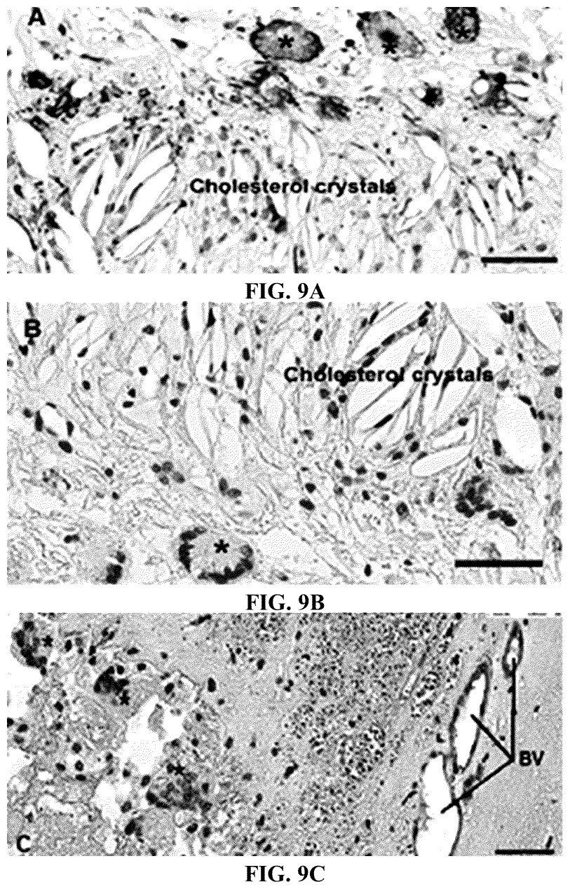

The multinucleated giant cells, formed by the adhesion and fusion of adjacent macrophages are a feature of atherosclerotic plaques in advanced stages of the disease. As illustrated in FIG. 9A the multinucleated giant cells express FSHR mainly on the cell surface. By contrast, the multinucleated giant cells were CD34-negative (FIG. 9C), a result that indicates that these cells do not originate from endothelial cells (Tokunaga et al., 1989), but from adhesion and fusion of adjacent M1-macrophages.

### Leukocytes

Leukocytes are a major component of advanced atherosclerotic lesions.

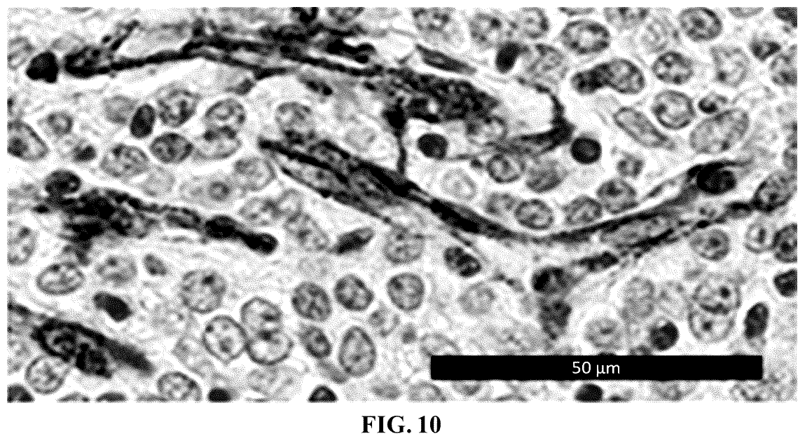

As illustrated in FIG. 10 these cells did not express FSHR.

### Production of a Mouse Model for Human Atherosclerosis: Transgenic ApoE-KO Mice Expressing the Human FSHR

Atherosclerosis-prone apolipoprotein E-deficient (ApoE-KO) mouse is a well-established model for the study of human atherosclerosis. Fed with an atherogenic diet these mice display poor lipoprotein clearance with subsequent accumulation of cholesterol ester-enriched particles in the blood, which promote the development of atherosclerotic plaques (Lo Sasso et al., 2016). Does the ApoE-KO mouse model express FSHR in their atherosclerotic plaques? To answer this question the FSHR-immunohistochemistry is the method to be used. Unfortunately, no specific antibodies for the mouse FSHR are commercially available. The inventor has already obtained five mouse anti-human FSHR monoclonal antibodies of very high affinity (WO2018/172078, Ghinea, 2018). However, due to their low affinity (EC50=0.5 μM) for murine FSHR these antibodies are not suitable to assess the expression of murine FSHR. Therefore, for this study the inventor has generated ApoE-KO mice expressing the human FSHR only in Sertoli cells of the testis and granulosa cells of the ovaries. By using a genotyping protocol (see Materials and Methods) the double homozygous ApoE-KO-hFSHR-KI mice have been selected to induce atherosclerotic lesions with high fed cholesterol diet.

### Immunohistochemical Detection of FSHR Expression in Atherosclerotic Plaques in ApoE-KO-hFSHR-KI Mice

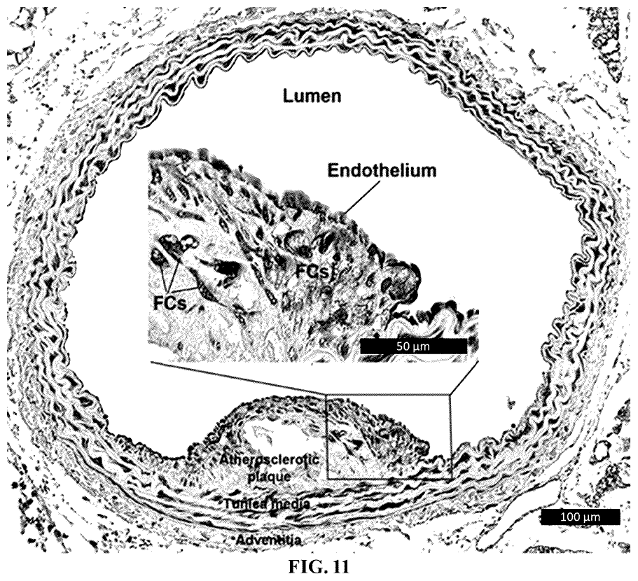

The expression of endothelial FSHR in atherosclerotic plaques in mice fed with a diet rich in cholesterol was analyzed in situ on paraffin embedded specimens of the aortic arch by standard peroxidase protocol as previously described (Radu et al., 2010). A strong signal for FSHR was detected on the arterial endothelium, smooth muscle cells, macrophages, and fat cells (FIG. 11).

Immunoelectron microscopy detection of FSHR expression in atherosclerotic plaques in ApoE-KO-hFSHR-KI mice

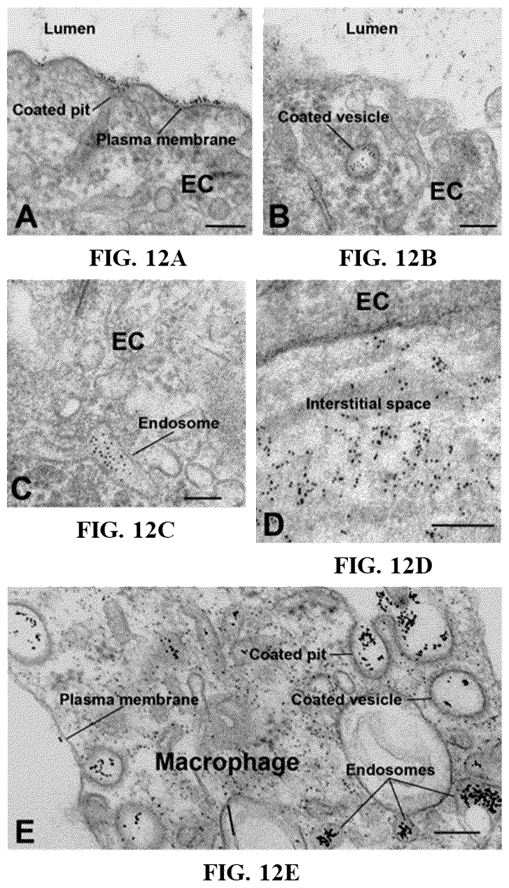

Although immunohistochemical data clearly indicate that FSHR is expressed in various cells associated with atherosclerotic plaques, the question is whether the antibodies raised against the extracellular domain of FSHR when injected in vivo have access to these FSHR-positive cells. To answer this important question, the inventor performed an immunoelectron study with living ApoE-KO-hFSHR-KI mice (an animal model for human atherosclerosis). As a tracer, he used the mouse anti-human FSHR monoclonal antibody 323 coupled to gold particles (visible by electron microscopic). The main advantage of this study is that the tracer is injected into living animals. Fixation of tissues is known “to freeze” the tracer in its location when the fixative is applied (Simionescu and Ghinea 1990). After 20 min of injection, the gold particles distributed on the plasma membrane (FIG. 12A) were internalized by clathrin-coated pits (FIG. 12B) and clathrin-coated vesicles (FIG. 12C) on the luminal aspect of the endothelium covering the atherosclerotic plaques. A large amount of gold labeled FSHR323 was noticed in the interstitial space of the atherosclerotic plaques (FIG. 12D), and internalized via clathrin-coated pits and clathrin-coated vesicles into the endosomes of macrophages present within the atherosclerotic plaques (FIG. 12E).

Taken together these results indicate: i) the in vivo experiments with anti-human FSHR monoclonal antibodies could be considered to be a proof-of-principle demonstration that the FSHR expressed on cells associated with the atherosclerotic plaques can be exploited clinically (i.e., by using humanized anti-hFSHR antibodies coupled to imaging and therapeutic agents), and ii) that the animal model used mimics a clinical application in humans.

TABLE 2
Characteristics of atherosclerotic plaque tissue samples used for quantification of FSHR-mRNA with the Nanostring method

| | | Cc° | Cc° | | | | | | % | | |
|---|---|---|---|---|---|---|---|---|---|---|---|
| | Preservation | (ng/μl) | (ng/μl) | V | Amount | RATIO | RATIO | | fragments | Kit or method used for | |
| Patient | method | Nanodrop | Agilent | (μl) | (ng) | 260/280 | 260/230 | RIN | >300 nt | Nucleid extraction | Comments |
| 1 | FF | 91.21 | 63 | 12 | 1094.52 | 1.93 | 1.35 | 2.1 | 53 | miRNeasy mini + cleanup | |
| 2 | FF | 42.18 | 14 | 12 | 506.16 | 1.75 | 0.15 | 1.9 | 4 | miRNeasy mini + cleanup | |
| 3 | FF | 245.7 | 206 | 12 | 2948.4 | 1.97 | 1.44 | 2.4 | 54 | miRNeasy mini + cleanup | |
| 4 | FF | 378.2 | 357 | 12 | 4538.4 | 1.97 | 0.89 | 6.9 | 60 | miRNeasy mini + cleanup | ok for Nanostring |
| 5 | FF | 163.1 | 115 | 12 | 1957.2 | 1.95 | 1.12 | N/A | 41 | miRNeasy mini + cleanup | ok for Nanostring |
| 6 | FF | 50.47 | 38 | 12 | 605.64 | 1.81 | 0.46 | 2.1 | 42 | miRNeasy mini + cleanup | |
| 7 | FF | 130 | 114 | 12 | 1560 | 1.93 | 1.4 | 2.6 | 20 | miRNeasy mini + cleanup | |
| 8 | FF | 12.54 | 4 | 12 | 150.48 | 1.55 | 0.38 | N/A | 52 | miRNeasy mini + cleanup | |
| 9 | FF | 765.2 | 803 | 12 | 9182.4 | 2.01 | 1.82 | 4.3 | 58 | miRNeasy mini + cleanup | ok for Nanostring |
| 10 | FF | 475.2 | 459 | 12 | 5702.4 | 1.95 | 1.08 | 6.2 | 72 | miRNeasy mini + cleanup | ok for Nanostring |

### CONCLUSIONS

FSHR-mRNA is present in human atherosclerotic plaques. Quantification of FSHR-mRNA by the Nanostring technology indicates the presence of 28+/−6.9 copies of the FSHR gene in the atherosclerotic plaque tissues in humans.

FSHR-protein is expressed in the endothelial cells of carotid and femoral arteries, and of intimal neovessels, M1-macrophages, M1-derived giant cells, and M1-foam cells associated with human atherosclerotic plaques are also FSHR-positive.

At advanced stages of the disease the human atherosclerotic plaques are rich in FSHR-positive blood neovessels and FSHR-negative lymphatic vessels.

Immature FSHR-positive vessels and hemorrhage are characteristic features of ruptured human atherosclerotic plaques.

M2-macrophages, M2-macrophage derived foam cells, T cells, and polymorphonuclear cells associated with human atherosclerotic plaques do not express FSHR.

Breading of ApoE-KO homozygous female mice with hFSHR-KI homozygous male mice allows production of a double homozygous ApoE-KO/hFSHR-KI transgenic mouse line as a model for human atherosclerosis.

Treatment of ApoE-KO/hFSHR-KI transgenic mice with a high cholesterol atherogenic diet induces the expression of FSHR on arterial endothelial cells, macrophages, and macrophage-derived foam cells associated with the atherosclerotic plaques at the level of the aortic roots and aortic arch.

Aortic endothelial cells covering the atherosclerotic plaques in ApoE-KO/hFSHR-KI mice are FSHR-positive. Immunoelectron microscopy with the use of anti-hFSHR 323 antibody-colloidal gold particles indicates that FSHR is mainly associated with the luminal plasma membrane and clathrin-coated pits.

### REFERENCES

DeMey J. In: Polak J M, Van Noorden S (Eds.) Immunocytochemistry: Practical Applications. In Pathology and Biology. Wright, Bristol, 1986, pp: 115-145.
Ghinea N. 2018. WO2018/172078 A1.
Knutsson et al., “Treatment with a GnRH receptor agonist, but not the GnRH receptor antagonist degarelix, induces atherosclerotic plaque instability in ApoE(−/−) mice”, Scientific Reports, May 18, 2016, 6:26220, doi: 10.1038/srep26220.
Lo Sasso et al., “The ApoE(−/−) mouse model: a suitable model to study cardiovascular and respiratory diseases in the context of cigarette smoke exposure and harm reduction”, Journal of Translational Medicine, May 20, 2016, 14(1): 146, doi: 10.1186/s12967-016-0901-1.
Möker et al., “Antibody Selection for Cancer Target Validation of FSH-Receptor in Immunohistochemical Settings”, Antibiotics, Oct. 18, 2017, 6(4): 15, doi: 10.3390/antib6040015.
Moore K J and Tabas I, “Macrophages in the pathogenesis of atherosclerosis”, Cell, Apr. 29, 2011, 145(3): 341-355, doi: 10.1016/j.cell.2011.04.005.
Ponikwicka-Tyszko et al., “Functional Expression of FSH Receptor in Endometriotic Lesions”, Journal of Clinical Endocrinology and Metabolism, July 2016, 101(7): 2905-14, doi: 10.1210/jc.2016-1014.
Radu et al., “Expression of follicle-stimulating hormone receptor in tumor blood vessels”, New England Journal of Medicine, Oct. 21, 2010, 363(17): 1621-30, doi: 10.1056/NEJMoa1001283.
Robin et al., “Follicle-Stimulating Hormone Receptor Expression in Endometriotic Lesions and the Associated Vasculature: An Immunohistochemical Study”, Reproductive Sciences, July 2016, 23(7): 885-91, doi: 10.1177/1933719115623647.
Sluimer J C and Daemen M J. “Novel concepts in atherogenesis: angiogenesis and hypoxia in atherosclerosis”, Journal of Pathology, May 2009, 218(1): 7-29, doi: 10.1002/path.2518.
Simionescu M and Ghinea N. The use of tracers in transport studies. In Models of lung diseases: Microscopy and structural methods. Edited by Joan Gil. Vol. 47, pages 359-408. Marcel Dekker, Inc./New York, Basel.
Simoni et al., “The follicle-stimulating hormone receptor: biochemistry, molecular biology, physiology, and pathophysiology”, Endocrine Reviews, December 1997, 18(6): 739-773, doi: 10.1210/edrv.18.6.0320.
Sprengel et al., “The testicular receptor for follicle stimulating hormone: structure and functional expression of cloned cDNA”, Molecular Endocrinology, April 1990, 4(4): 525-530, doi: 10.1210/mend-4-4-525.
Stilley et al., “FSH receptor (FSHR) expression in human extragonadal reproductive tissues and the developing placenta, and the impact of its deletion on pregnancy in mice”, Biology of Reproduction, September 2014, 91(3): 74, doi: 10.1095/biolreprod.
Tokunaga et al., “Atherosclerosis- and age-related multinucleated variant endothelial cells in primary culture from human aorta”, The American Journal of Pathology, December 1989, 135(6): 967-76.
Vannier et al., “Anti-human FSH receptor monoclonal antibodies: immunochemical and immunocytochemical characterization of the receptor” Biochemistry, Feb. 6, 1996, 35(5): 1358-66, doi: 10.1021/bi952290f.
Virmani et al., “Atherosclerotic plaque progression and vulnerability to rupture: angiogenesis as a source of intraplaque hemorrhage”, Arteriosclerosis, Thrombosis, and Vascular Biology, October 2005, 25(10): 2054-61, doi: 10.1161/01.ATV.0000178991.71605.18.
Vu Hai et al., “Receptor-mediated transcytosis of follicle-stimulating hormone through the rat testicular microvasculature”, Biology of the Cell, March 2004, 96(2): 133-44, doi: 10.1016/j.biolcel.2003.11.008.

1-13. (canceled)

14. A method for imaging or diagnosing a condition associated with an atherosclerotic lesion, wherein said method comprises:
a) providing an imaging agent comprising a follicle-stimulating hormone receptor (FSHR) ligand;
b) administering a patient with said imaging agent;
c) collecting imaging data in said patient;
d) detecting atherosclerotic plaques and imaging or diagnosing the condition associated with the atherosclerotic plaques.

15. The method according to claim 14, wherein the FSHR ligand is detectably labeled FSH.

16. The method according to claim 14, wherein the FSHR ligand is a detectably labeled FSHR-binding aptamer.

17. The method according to claim 14, wherein the FSHR ligand is a detectably labeled anti-FSHR antibody or antigen-binding fragment thereof.

18. The method according to claim 14, wherein the FSHR ligand is a detectably labeled anti-FSHR antibody or antigen-binding fragment thereof comprising:
(a) a variable region of the heavy chain
comprising the following CDRs:
VH-CDR1:
(SEQ ID NO: 1)
QFYVG,
VH-CDR2:
(SEQ ID NO: 6)
EIFPRTGNTNYNEKFKG,
VH-CDR3:
(SEQ ID NO: 11)
GPTASGYAMDY,
and
a variable region of the light chain
comprising the following CDRs:
VL-CDR1:
(SEQ ID NO: 12)
RSSQSIVHRNGNTYLE,
VL-CDR2:
(SEQ ID NO: 13)
KVSNRFS,
and
VL-CDR3:
(SEQ ID NO: 14)
FQGSHVPFT;
or
(b) a variable region of the heavy chain
comprising the following CDRs:
VH-CDR1:
(SEQ ID NO: 2)
RQWVI,
VH-CDR2:
(SEQ ID NO: 7)
EILPRNGNTNYNEKFKG,
VH-CDR3:
(SEQ ID NO: 11)
GPTASGYAMDY,
and
a variable region of the light chain
comprising the following CDRs:
VL-CDR1:
(SEQ ID NO: 12)
RSSQSIVHRNGNTYLE,
VL-CDR2:
(SEQ ID NO: 13)
KVSNRFS,
and
VL-CDR3:
(SEQ ID NO: 14)
FQGSHVPFT;
or
(c) a variable region of the heavy chain
comprising the following CDRs:
VH-CDR1:
(SEQ ID NO: 3)
KQWLL,
VH-CDR2:
(SEQ ID NO: 8)
EIFPRNGNTNYNEKFKG,
VH-CDR3:
(SEQ ID NO: 11)
GPTASGYAMDY,
and
a variable region of the light chain
comprising the following CDRs:
VL-CDR1:
(SEQ ID NO: 12)
RSSQSIVHRNGNTYLE,
VL-CDR2:
(SEQ ID NO: 13)
KVSNRFS,
and
VL-CDR3:
(SEQ ID NO: 14)
FQGSHVPFT;
or
(d) a variable region of the heavy chain
comprising the following CDRs:
VH-CDR1:
(SEQ ID NO: 4)
RSWIL,
VH-CDR2:
(SEQ ID NO: 9)
EIYPQNQNTNYNEKFKG,
VH-CDR3:
(SEQ ID NO: 11)
GPTASGYAMDY,
and
a variable region of the light chain
comprising the following CDRs:
VL-CDR1:
(SEQ ID NO: 12)
RSSQSIVHRNGNTYLE,
VL-CDR2:
(SEQ ID NO: 13)
KVSNRFS,
and
VL-CDR3:
(SEQ ID NO: 14)
FQGSHVPFT;
or
(e) a variable region of the heavy chain
comprising the following CDRs:
VH-CDR1:
(SEQ ID NO: 5)
KYWTQ,
VH-CDR2:
(SEQ ID NO: 10)
EIYPRNGNTNYNEKFKG,
VH-CDR3:
(SEQ ID NO: 11)
GPTASGYAMDY,
and
a variable region of the light chain
comprising the following CDRs:
VL-CDR1:
(SEQ ID NO: 12)
RSSQSIVHRNGNTYLE,
VL-CDR2:
(SEQ ID NO: 13)
KVSNRFS,
and
VL-CDR3:
(SEQ ID NO: 14)
FQGSHVPFT.

19. The method according to claim 14, wherein the FSHR ligand is a detectably labeled anti-FSHR antibody or antigen-binding fragment thereof comprising:
(a) a heavy chain comprising the amino acid
sequence:
(SEQ ID NO: 15)
QVQLQQSGAELMKPGASVKISCKATGYTFSQFYVGWVKQRPGHGLEWIGE
IFPRTGNTNYNEKFKGKATFTADTSSSTAYMQLSSLTSEDSAVYYCARGP
TASGYAMDYWGQGTSVTVSS,
and
a light chain comprising the amino acid sequence:
(SEQ ID NO: 20)
DVLMTQTPLSLPVSLGDQASISCRSSQSIVHRNGNTYLEWYLQKPGQSPK
LLIYKVSNRFSGVPDRFSGSGSGTDFTLKIIRVEAEDLGVYYCFQGSHVP
FTFGSGTKLEIK;
or
(b) a heavy chain comprising the amino acid sequence:
(SEQ ID NO: 16)
QVQLQQSGAELMKPGASVKISCKATGYTFSRQWVIWVKQRPGHGLEWIGE
ILPRNGNTNYNEKFKGKATFTADTSSSTAYMQLSSLTSEDSAVYYCARGP
TASGYAMDYWGQGTSVTVSS,
and
a light chain comprising the amino acid sequence:
(SEQ ID NO: 20)
DVLMTQTPLSLPVSLGDQASISCRSSQSIVHRNGNTYLEWYLQKPGQSPK
LLIYKVSNRFSGVPDRFSGSGSGTDFTLKIIRVEAEDLGVYYCFQGSHVP
FTFGSGTKLEIK;
or
(c) a heavy chain comprising the amino acid
sequence:
(SEQ ID NO: 17)
QVQLQQSGAELMKPGASVKISCKATGYTFSKQWLLWVKQRPGHGLEWIGEI
FPRNGNTNYNEKFKGKATFTADTSSSTAYMQLSSLTSEDSAVYYCARGPTA
SGYAMDYWGQGTSVTVSS,
and
a light chain comprising the amino acid
sequence:
(SEQ ID NO: 20)
DVLMTQTPLSLPVSLGDQASISCRSSQSIVHRNGNTYLEWYLQKPGQSPKL
LIYKVSNRFSGVPDRFSGSGSGTDFTLKIIRVEAEDLGVYYCFQGSHVPFT
FGSGTKLEIK;
or
(d) a heavy chain comprising the amino acid
sequence:
(SEQ ID NO: 18)
QVQLQQSGAELMKPGASVKISCKATGYTFSRSWILWVKQRPGHGLEWIGE
IYPQNQNTNYNEKFKGKATFTADTSSSTAYMQLSSLTSEDSAVYYCARGP
TASGYAMDYWGQGTSVTVSS,
and
a light chain comprising the amino acid
sequence:
(SEQ ID NO: 20)
DVLMTQTPLSLPVSLGDQASISCRSSQSIVHRNGNTYLEWYLQKPGQSPKL
LIYKVSNRFSGVPDRFSGSGSGTDFTLKIIRVEAEDLGVYYCFQGSHVPFT
FGSGTKLEIK;
or
(e) a heavy chain comprising the amino acid
sequence:
(SEQ ID NO: 19)
QVQLQQSGAELMKPGASVKISCKATGYTFSKYWTQWVKQRPGHGLEWIGEI
YPRNGNTNYNEKFKGKATFTADTSSSTAYMQLSSLTSEDSAVYYCARGPTA
SGYAMDYWGQGTSVTVSS,
and
a light chain comprising the amino acid
sequence:
(SEQ ID NO: 20)
DVLMTQTPLSLPVSLGDQASISCRSSQSIVHRNGNTYLEWYLQKPGQSPKL
LIYKVSNRFSGVPDRFSGSGSGTDFTLKIIRVEAEDLGVYYCFQGSHVPFT
FGSGTKLEIK.

20. The method according to claim 14, wherein the FSHR ligand is in form of an imaging composition for intravenous administration.

21. A method for collecting imaging data on the atherosclerotic plaques, wherein said method comprises:
a) providing an imaging agent comprising a detectably labeled FSHR ligand;
b) administering said imaging agent to a patient; and
c) collecting imaging data in said patient.

22. A method for treating atherosclerosis comprising administering a FSHR blocking agent in a subject in need thereof.

23. The method according to claim 22, wherein said FSHR blocking agent is an agent that blocks FSHR expression or intracellular signaling in atherosclerotic plaques.

24. The method according to claim 22, wherein said FSHR blocking agent is an anti-FSHR antibody or antigen-binding fragment thereof that prevents FSH induced signaling in atherosclerotic plaques.

25. The method according to claim 22, wherein the FSHR blocking agent is an anti-FSHR antibody or antigen-binding fragment thereof comprising:
(a) a variable region of the heavy chain
comprising the following CDRs:
VH-CDR1:
(SEQ ID NO: 1)
QFYVG,
VH-CDR2:
(SEQ ID NO: 6)
EIFPRTGNTNYNEKFKG,
VH-CDR3:
(SEQ ID NO: 11)
GPTASGYAMDY,
and
a variable region of the light chain
comprising the following CDRs:
VL-CDR1:
(SEQ ID NO: 12)
RSSQSIVHRNGNTYLE,
VL-CDR2:
(SEQ ID NO: 13)
KVSNRFS,
and
VL-CDR3:
(SEQ ID NO: 14)
FQGSHVPFT;
or
(b) a variable region of the heavy chain
comprising the following CDRs:
VH-CDR1:
(SEQ ID NO: 2)
RQWVI,
VH-CDR2:
(SEQ ID NO: 7)
EILPRNGNTNYNEKFKG,
VH-CDR3:
(SEQ ID NO: 11)
GPTASGYAMDY,
and
a variable region of the light chain
comprising the following CDRs:
VL-CDR1:
(SEQ ID NO: 12)
RSSQSIVHRNGNTYLE,
VL-CDR2:
(SEQ ID NO: 13)
KVSNRFS,
and
VL-CDR3:
(SEQ ID NO: 14)
FQGSHVPFT;
or
(c) a variable region of the heavy chain
comprising the following CDRs:
VH-CDR1:
(SEQ ID NO: 3)
KQWLL,
VH-CDR2:
(SEQ ID NO: 8)
EIFPRNGNTNYNEKFKG,
VH-CDR3:
(SEQ ID NO: 11)
GPTASGYAMDY,
and
a variable region of the light chain
comprising the following CDRs:
VL-CDR1:
(SEQ ID NO: 12)
RSSQSIVHRNGNTYLE,
VL-CDR2:
(SEQ ID NO: 13)
KVSNRFS,
and
VL-CDR3:
(SEQ ID NO: 14)
FQGSHVPFT;
or
(d) a variable region of the heavy chain
comprising the following CDRs:
VH-CDR1:
(SEQ ID NO: 4)
RSWIL,
VH-CDR2:
(SEQ ID NO: 9)
EIYPQNQNTNYNEKFKG,
VH-CDR3:
(SEQ ID NO: 11)
GPTASGYAMDY,
and
a variable region of the light chain
comprising the following CDRs:
VL-CDR1:
(SEQ ID NO: 12)
RSSQSIVHRNGNTYLE,
VL-CDR2:
(SEQ ID NO: 13)
KVSNRFS,
and
VL-CDR3:
(SEQ ID NO: 14)
FQGSHVPFT;
or
(e) a variable region of the heavy chain
comprising the following CDRs:
VH-CDR1:
(SEQ ID NO: 5)
KYWTQ,
VH-CDR2:
(SEQ ID NO: 10)
EIYPRNGNTNYNEKFKG,
VH-CDR3:
(SEQ ID NO: 11)
GPTASGYAMDY,
and
a variable region of the light chain
comprising the following CDRs:
VL-CDR1:
(SEQ ID NO: 12)
RSSQSIVHRNGNTYLE,
VL-CDR2:
(SEQ ID NO: 13)
KVSNRFS,
and
VL-CDR3:
(SEQ ID NO: 14)
FQGSHVPFT.

26. The method according to claim 22, wherein the FSHR blocking agent is an anti-FSHR antibody or antigen-binding fragment thereof comprising:
(a) a heavy chain comprising the amino acid
sequence:
(SEQ ID NO: 15)
QVQLQQSGAELMKPGASVKISCKATGYTFSQFYVGWVKORPGHGLEWIGE
IFPRTGNTNYNEKFKGKATFTADTSSSTAYMQLSSLTSEDSAVYYCARGP
TASGYAMDYWGQGTSVTVSS,
and
a light chain comprising the amino acid
sequence:
(SEQ ID NO: 20)
DVLMTQTPLSLPVSLGDQASISCRSSQSIVHRNGNTYLEWYLQKPGQSPK
LLIYKVSNRFSGVPDRFSGSGSGTDFTLKIIRVEAEDLGVYYCFQGSHVP
FTFGSGTKLEIK;
or
(b) a heavy chain comprising the amino acid
sequence:
(SEQ ID NO: 16)
QVQLQQSGAELMKPGASVKISCKATGYTFSRQWVIWVKQRPGHGLEWIGE
ILPRNGNTNYNEKFKGKATFTADTSSSTAYMQLSSLTSEDSAVYYCARGP
TASGYAMDYWGQGTSVTVSS,
and
a light chain comprising the amino acid
sequence:
(SEQ ID NO: 20)
DVLMTQTPLSLPVSLGDQASISCRSSQSIVHRNGNTYLEWYLQKPGQSPKL
LIYKVSNRFSGVPDRFSGSGSGTDFTLKIIRVEAEDLGVYYCFQGSHVPFT
FGSGTKLEIK;
or
(c) a heavy chain comprising the amino acid
sequence:
(SEQ ID NO: 17)
QVQLQQSGAELMKPGASVKISCKATGYTFSKQWLLWVKQRPGHGLEWIGE
IFPRNGNTNYNEKFKGKATFTADTSSSTAYMQLSSLTSEDSAVYYCARGP
TASGYAMDYWGQGTSVTVSS,
and
a light chain comprising the amino acid
sequence:
(SEQ ID NO: 20)
DVLMTQTPLSLPVSLGDQASISCRSSQSIVHRNGNTYLEWYLQKPGQSPKL
LIYKVSNRFSGVPDRFSGSGSGTDFTLKIIRVEAEDLGVYYCFQGSHVPFT
FGSGTKLEIK;
or
(d) a
heavy chain comprising the amino acid sequence:
(SEQ ID NO: 18)
QVQLQQSGAELMKPGASVKISCKATGYTFSRSWILWVKQRPGHGLEWIGE
IYPQNQNTNYNEKFKGKATFTADTSSSTAYMQLSSLTSEDSAVYYCARGP
TASGYAMDYWGQGTSVTVSS,
and
a light chain comprising the amino acid
sequence:
(SEQ ID NO: 20)
DVLMTQTPLSLPVSLGDQASISCRSSQSIVHRNGNTYLEWYLQKPGQSPKL
LIYKVSNRFSGVPDRFSGSGSGTDFTLKIIRVEAEDLGVYYCFQGSHVPFT
FGSGTKLEIK;
or
(e) a heavy chain comprising the amino acid
sequence:
(SEQ ID NO: 19)
QVQLQQSGAELMKPGASVKISCKATGYTFSKYWTQWVKQRPGHGLEWIGEI
YPRNGNTNYNEKFKGKATFTADTSSSTAYMQLSSLTSEDSAVYYCARGPTA
SGYAMDYWGQGTSVTVSS,
and
a light chain comprising the amino acid
sequence:
(SEQ ID NO: 20)
DVLMTQTPLSLPVSLGDQASISCRSSQSIVHRNGNTYLEWYLQKPGQSPKL
LIYKVSNRFSGVPDRFSGSGSGTDFTLKIIRVEAEDLGVYYCFQGSHVPFT
FGSGTKLEIK.
